(19) United States
(12) Patent Application Publication
(10) Pub. No.: US 2022/0054493 A1
(43) Pub. Date: Feb. 24, 2022

(54) INHIBITORS OF NGAL PROTEIN

(71) Applicants: INSERM (INSTITUT NATIONAL DE LA SANTÉ ET DE LA RECHERCHE MÉDICALE), Paris (FR); UNIVERSITE DE ROUEN-NORMANDIE, Mont-Saint-Aignan (FR); GREENPHARMA, Orleans (FR); UNIVERSITE DE PARIS, Paris (FR); SORBONNE UNIVERSITÉ, Paris (FR)

(72) Inventors: Frédéric JAISSER, Paris (FR); Ernesto MARTINEZ-MARTINEZ, Madrid (ES); Paul MULDER, Rouen (FR); Antoine OUVRARD-PASCAUD, Rouen (FR); Philippe BERNARD, Orleans (FR); Quoc Tuan DO, Orleans (FR)

(21) Appl. No.: 17/434,535

(22) PCT Filed: Mar. 6, 2020
(86) PCT No.: PCT/EP2020/056077

§ 371 Date: Aug. 27, 2021

(30) Foreign Application Priority Data
Mar. 6, 2019 (EP) 19305259.4
May 27, 2019 (EP) 19305665.2

(51) Int. Cl. A61K 31/519; A61K 31/4245; A61K 31/415

(52) U.S. Cl. CPC A61K 31/519; A61K 31/415; A61K 31/4245

(57) ABSTRACT

This invention relates to compounds that are inhibitors of NGAL activity, and applications thereof.

### INTRODUCTION

This invention relates to compounds that are inhibitors of NGAL activity, and applications thereof.

### BACKGROUND OF THE INVENTION

Neutrophil Gelatinase-Associated Lipocalin (NGAL) (also known as lipocalin-2, oncogene 24p3, siderocalin or uterocalin) is a small circulating protein induced in a wide variety of pathological situations. Initially, NGAL has been identified in mature neutrophil granules. It has also been discovered that NGAL was expressed in the kidney, prostate, and epithelia of the respiratory and alimentary tracts. In particular, NGAL is expressed in many other cell types such as in renal, endothelial, liver and smooth muscle cells, but also in cardiomyocytes, neurons and in different populations of immune cells such as macrophages and dendritic cells. Studies have highlighted the implication of NGAL in renal injuries or its role as inflammation biomarker. Other researches demonstrated that NGAL has a link with growth factor, a role in iron trafficking, chemotactic and bacteriostatic effects, as well as activities such as differentiation, proliferation and inflammation. For example, NGAL participates to the epithelial-mesenchymal transition in vivo in a pulmonary adenocarcinoma model and in vitro in prostate and breast cancer cells. In these models, NGAL promoted the motility, invasiveness and metastatic capacities of cancer cells. NGAL is also involved in cardiovascular, metabolic and renal diseases. For example, gene inactivation in mice blunted the pathophysiological consequences of cardiovascular (myocardial infarction or ischemia), renal (subtotal nephrectomy) or metabolic (High Fat Diet) challenges.

There is thus a need to found NGAL inhibitors which can be used as therapeutic agents since NGAL protein is involved in various diseases.

The present inventors have found compounds which can be used as NGAL inhibitors and thus can be used in the treatment and/or prevention of NGAL involved diseases.

### SUMMARY OF THE INVENTION

The present invention relates to the use of a compound of general formula (I) as a NGAL inhibitor:

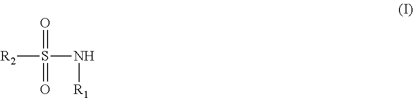

wherein

R₁ represents:
(CH₂)ₙ₁-pyrazole optionally substituted by an aryl group or a pyridinyl group,
wherein n₁ represent an integer between 0 and 1,
(CH₂)ₙ₂-aryl, said aryl being optionally substituted by one or more:
   pyrazolyl groups,
   —CH₂-pyrazolyl groups,
   thiophenyl groups,
   pyridinyl groups or
   —CH₂-piperazinyl groups optionally substituted by one or more ethyl groups,
   phenyl groups optionally substituted by one or more —(CH₂)n₃-N(—CH₃)(—CH₃),
wherein n₂ and n₃ each independently represent an integer between 0 and 1, or
—C(═O)—N(H)—R₃ wherein R₃ represents a cyclohexyl group;

R2 represents an aryl group optionally substituted by one or more:
—C(═O)—R₄ wherein R₄ represents a methyl group;
—C≡N; or
—NH₂.

The present invention also relates to the use of a compound of general formula (II) as a NGAL inhibitor:

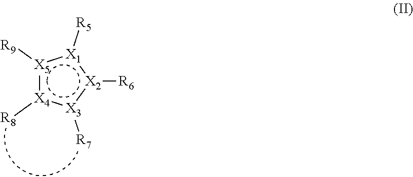

wherein:

X₁ represents a nitrogen atom, or a carbon atom;

X₂ represents a carbon atom, or a CH group;

X₃ represents a nitrogen atom;

X₄ represents an oxygen atom, or a carbon atom;

X₅ represents a carbon atom, or a nitrogen atom;

R₅ represents:
(CH₂)n₄-N(—C₂H₅)(—C₂H₅),
wherein n₄ represents an integer between 0 and 2,
—CH₂—S—R₁₀,
—S—CH₂—R₁₁, or
—C(═O)—N(H)—R₁₂,
wherein R₁₀ R₁₁ and R₁₂ each independently represent an heterocycle of general formula (IV)

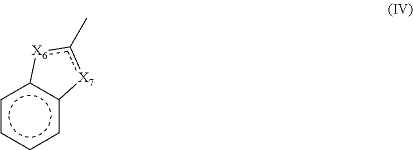

wherein X₆ represents a nitrogen atom, a —NH group, an oxygen atom, or a sulphur atom,
   X₇ represents an oxygen atom or a nitrogen atom,
said heterocycle being optionally substituted by one or more —S(═O)(═O)(—CH₂H₅), —C(═O)Me, halogeno atoms, trifluoromethyl groups, cyano groups, or nitro groups;

R₆ represents a phenyl group, a lone pair, an oxygen atom, or an halogeno atom;

R₉ represents a ═NH group, or a lone pair;

R₇ and R₈ represent a lone pair or R₈—X₄—X₃—R₇ optionally form a six membered ring heterocycle, said heterocycle being optionally substituted by one or more methyl groups, preferably by two methyl groups.

The present invention also relates to the use of a compound of general formula (III) as a NGAL inhibitor:

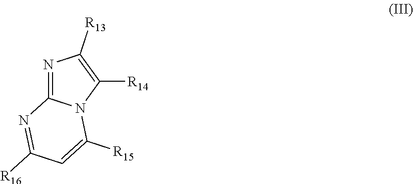

wherein

R₁₃ represents
—CH₂—S—R₁₇,
—S—CH₂—R₁₈, or
—C(═O)—N(H)—R₁₉,
wherein R₁₇, R₁₈ and R₁₉ each independently represent an heterocycle of general formula (V)

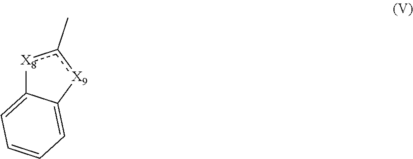

wherein X₈ represents a nitrogen atom, a —NH group, an oxygen atom, or a sulphur atom,
   X₉ represents an oxygen atom or a nitrogen atom, said heterocycle being optionally substituted by one or more —S(═O)(═O)(—CH₂H₅), halogeno atoms or nitro groups;

R₁₄ represents a lone pair, a hydrogen atom or an halogen atom;

R₁₅ represents a methyl group, a hydrogen atom or a lone pair; and

R₁₆ represents a methyl group, a hydrogen atom or a lone pair.

### DETAILED DESCRIPTION OF THE PREFERRED EMBODIMENTS

As used herein, the term “NGAL” has its general meaning in the art and refers to the Neutrophil Gelatinase-Associated Lipocalin as described in Schmidt-Ott K M. et al. (2007) (Schmidt-Ott K M, Mori K, Li J Y, Kalandadze A, Cohen D J, Devarajan P, Barasch J. Dual action of neutrophil gelatinase-associated lipocalin. J Am Soc Nephrol. 2007 February; 18(2):407-13. Epub 2007 Jan. 17. Review.). NGAL was shown to exist both as a 25-kDa monomer and a 45-kDa disulfide-linked homodimer, and it may also be covalently complexed with neutrophil gelatinase (also known as matrix metalloproteinase 9, MMP-9) via an intermolecular disulphide bridge as a 135-kDa heterodimeric form.

An “inhibitor of NGAL activity” has its general meaning in the art, and refers to a compound (natural or not) which has the capability of reducing or suppressing the activity of NGAL. For example the compound may block the interaction of NGAL with the NGAL binding ligands, or may bind to NGAL in manner that NGAL losses its capacity to interact with its receptors (for example 24p3R, megalin, SLC22A17 Solute carrier family 22 member 17) or with other proteins (for example the metalloprotease MMP9), thereby modifying the NGAL-mediated signalling.

In the present invention, the wording “inhibitor of NGAL activity” and “NGAL inhibitor” have the same meaning.

Typically, the inhibitory activity of an NGAL inhibitor as defined in the present invention is evaluated on the NGAL-induced IL6 secretion. In particular, an NGAL inhibitor as defined in the present invention is able to prevent the increase of IL-6 production and secretion induced by NGAL in primary culture of human cardiac fibroblasts.

Without wishing to be bound by theory, the “NGAL inhibitor” as defined in the present invention may refer to an inhibitor which inhibits the interaction of the NGAL with its receptor based on NGAL surface cavities. These hot-spots may be putative protein-protein contact interfaces between NGAL and its cognate receptor and the NGAL inhibitor may disrupt the corresponding interactions.

### NGAL Inhibitors of Formula (I)

The present invention relates to the use of a compound of general formula (I) as a NGAL inhibitor:

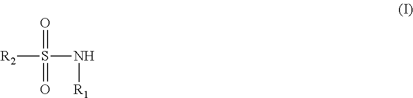

wherein:

R₁ represents:
(CH₂)ₙ₁-pyrazole optionally substituted by an aryl group or a pyridinyl group,
wherein n₁ represent an integer between 0 and 1,
(CH₂)ₙ₂-aryl, said aryl being optionally substituted by one or more:
   pyrazolyl groups,
   —CH₂-pyrazolyl groups,
   thiophenyl groups,
   pyridinyl groups or
   —CH₂-piperazinyl groups optionally substituted by one or more ethyl groups,
   phenyl groups optionally substituted by one or more —(CH₂)n₃-N(—CH₃)(—CH₃),
wherein n₂ and n₃ each independently represent an integer between 0 and 1, or
—C(═O)—N(H)—R₃ wherein R₃ represents a cyclohexyl group;

R2 represents an aryl group optionally substituted by one or more:
—C(═O)—R₄ wherein R₄ represents a methyl group;
—C≡N; or
—NH₂.

In a particular embodiment, a compound wherein R₁ represents (CH₂)ₙ₂-aryl wherein n₂ is 0 and R₂ represents an aryl group substituted by —C≡N is excluded.

In a particular embodiment, a compound wherein R₂ represents an aryl group substituted by —NH₂ and R₁ represents (CH₂)n₂-aryl wherein n₂ is 1 is excluded.

In a particular embodiment, a compound wherein R₁ represents (CH₂)ₙ₁-pyrazole substituted by a pyridinyl group wherein n₁ is 0 and R₂ represents an aryl group substituted by —C≡N is excluded.

In a particular embodiment, a compound wherein R₁ represents (CH₂)ₙ₂-aryl wherein n₂ is 0 or 1 and R₂ represents an aryl group, an aryl group substituted by —C≡N or an aryl group substituted by —NH₂ is excluded.

In a particular embodiment, a compound wherein R₁ represents (CH₂)n₂-aryl wherein n₂ is 0 substituted by a pyrazolyl group and R₂ represents an aryl group substituted by —C(═O)—R₄ wherein R₄ represents a methyl group is excluded.

In a particular embodiment, a compound wherein R₁ represents —C(═O)—N(H)—R₃ wherein R₃ represents a cyclohexyl group and R₂ represents an aryl group substituted by —C(═O)—R₄ wherein R₄ represents a methyl group is excluded.

In a particular embodiment, a compound wherein R₁ represents (CH₂)ₙ₂-aryl wherein n₂ is 0 and R₂ represents an aryl group substituted by —C(═O)—R₄ wherein R₄ represents a methyl group is excluded.

In a particular embodiment, the compound of formula (I) is selected from the group consisting of:

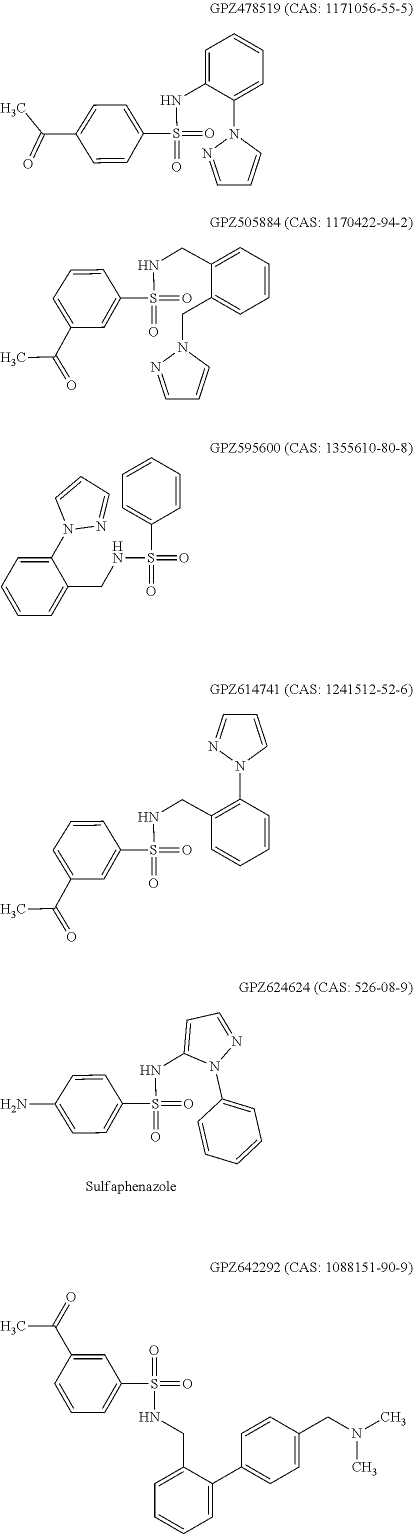

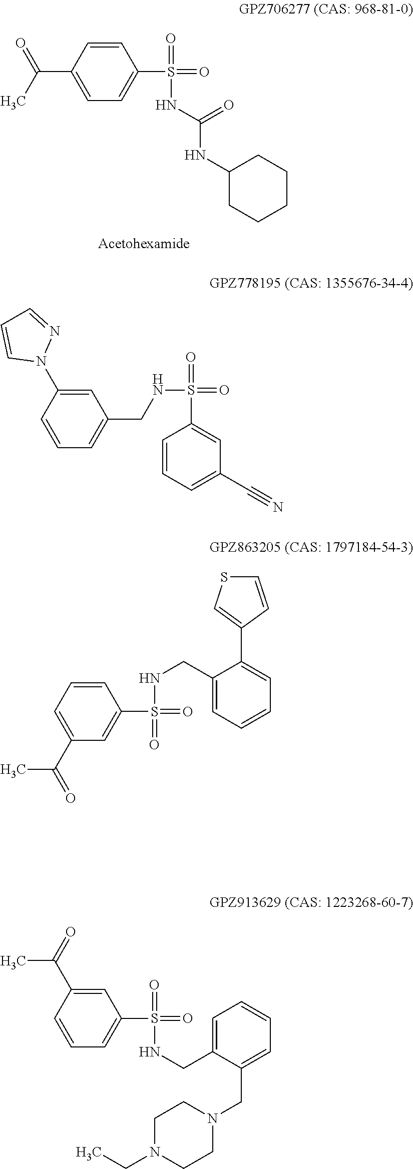 [SMILES: CC(=O)c1ccc(S(=O)(=O)NC(=O)NC2CCCCC2)cc1 ; N#Cc1cccc(S(=O)(=O)NCc2cccc(-n3cccn3)c2)c1 ; CC(=O)c1cccc(S(=O)(=O)NCc2ccccc2-c2ccsc2)c1 ; CCN1CCN(Cc2ccccc2CNS(=O)(=O)c2cccc(C(C)=O)c2)CC1]

In a particular embodiment, the compound of formula (I) is selected from the group consisting of:

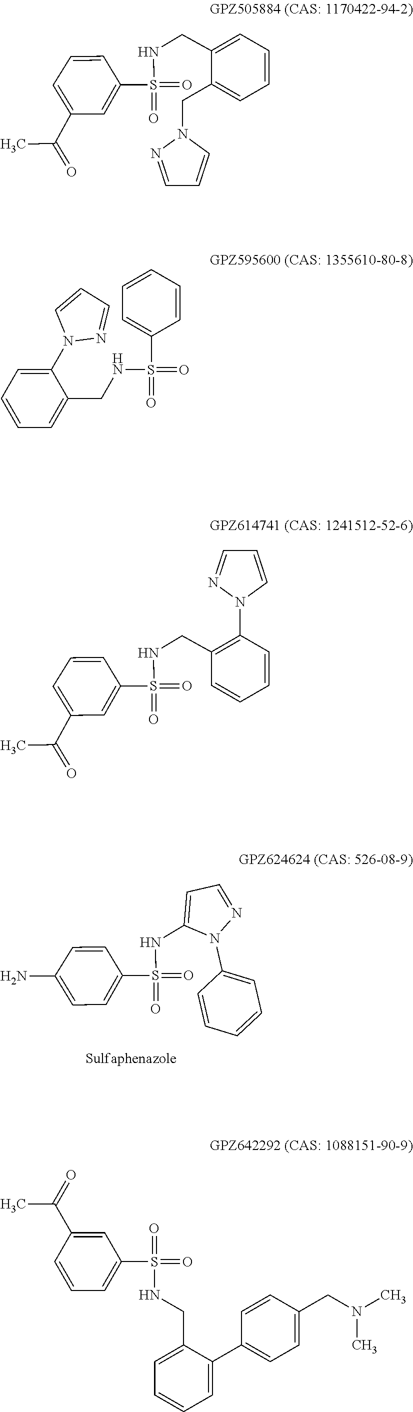 [SMILES: CC(=O)c1cccc(S(=O)(=O)NCc2ccccc2Cn2cccn2)c1 ; O=S(=O)(NCc1ccccc1-n1cccn1)c1ccccc1 ; CC(=O)c1cccc(S(=O)(=O)NCc2ccccc2-n2cccn2)c1 ; Nc1ccc(S(=O)(=O)Nc2ccnn2-c2ccccc2)cc1 ; CC(=O)c1cccc(S(=O)(=O)NCc2ccccc2-c2ccc(CN(C)C)cc2)c1]

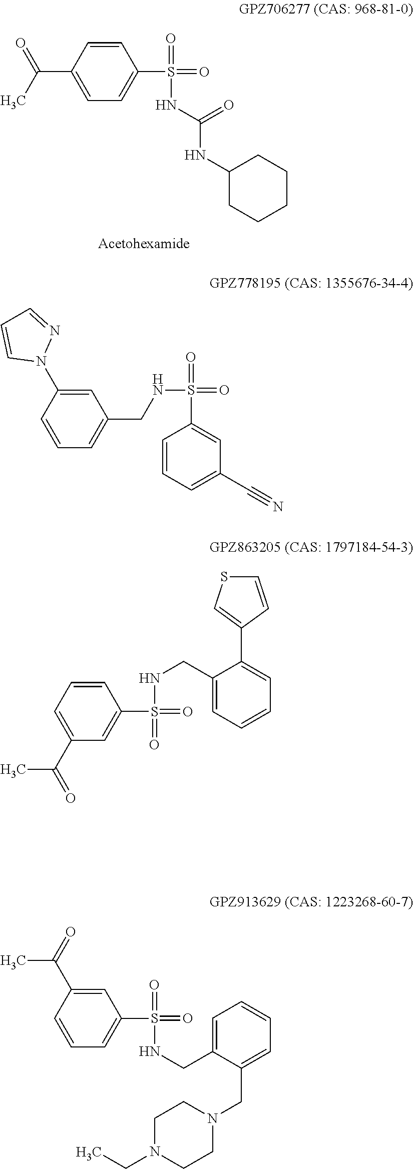 [SMILES: CC(=O)c1ccc(S(=O)(=O)NC(=O)NC2CCCCC2)cc1 ; N#Cc1cccc(S(=O)(=O)NCc2cccc(-n3cccn3)c2)c1 ; CC(=O)c1cccc(S(=O)(=O)NCc2ccccc2-c2ccsc2)c1 ; CCN1CCN(Cc2ccccc2CNS(=O)(=O)c2cccc(C(C)=O)c2)CC1]

The present invention also relates to the use of a compound of general formula (I) as a NGAL inhibitor:

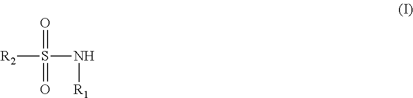

wherein

R₁ represents:
(CH₂)ₙ₂-aryl, said aryl being optionally substituted by one or more:
   pyrazolyl groups,
   —CH₂-pyrazolyl groups,
   thiophenyl groups,
   pyridinyl groups, or
   —CH₂-piperazinyl groups optionally substituted by one or more ethyl groups,
   phenyl groups optionally substituted by one or more —(CH₂)n₃-N(—CH₃)(—CH₃),
wherein n₂ and n₃ each independently represent an integer between 0 and 1.

R₂ represents an aryl group optionally substituted by one or more:
—C(═O)—R₄ wherein R₄ represents a methyl group; or
—C≡N.

In a particular embodiment, a compound wherein R₁ represents (CH₂)ₙ₂-aryl wherein n₂ is 0 and R₂ represents an aryl group substituted by —C≡N is excluded.

In a particular embodiment, a compound wherein R₂ represents an aryl group substituted by —NH₂ and R₁ represents (CH₂)ₙ₂-aryl wherein n₂ is 1 is excluded.

In a particular embodiment, a compound wherein R₁ represents (CH₂)ₙ₁-pyrazole substituted by a pyridinyl group wherein n₁ is 0 and R₂ represents an aryl group substituted by —C≡N is excluded.

In a particular embodiment, a compound wherein R₁ represents (CH₂)ₙ₂-aryl wherein n₂ is 0 or 1 and R₂ represents an aryl group, an aryl group substituted by —C≡N or an aryl group substituted by —NH₂ is excluded.

In a particular embodiment, a compound wherein R₁ represents (CH₂)ₙ₂-aryl wherein n₂ is 0 substituted by a pyrazolyl group and R₂ represents an aryl group substituted by —C(═O)—R₄ wherein R₄ represents a methyl group is excluded.

In a particular embodiment, a compound wherein R₁ represents (CH₂)ₙ₂-aryl wherein n₂ is 0 and R₂ represents an aryl group substituted by —C(═O)—R₄ wherein R₄ represents a methyl group is excluded.

In a particular embodiment, the compound of general formula (I) is selected from the group consisting of:

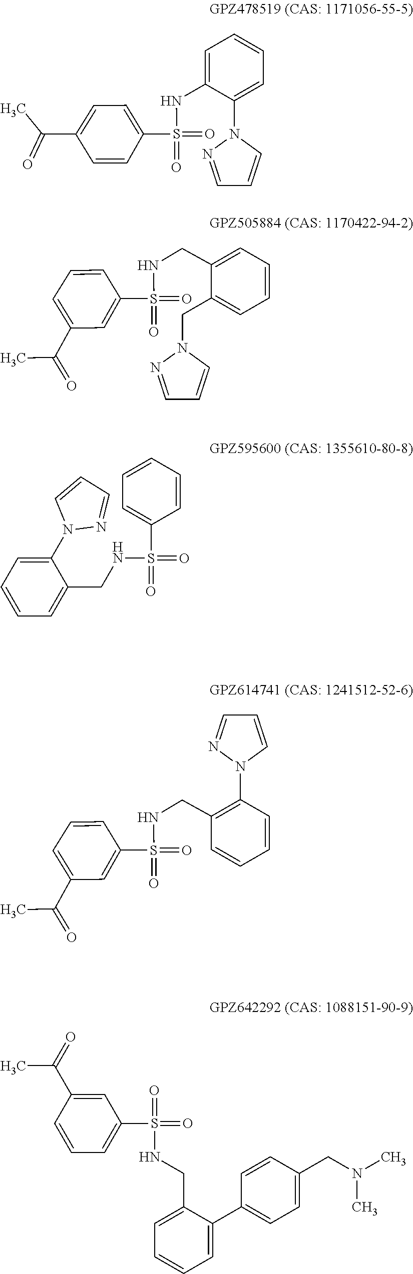

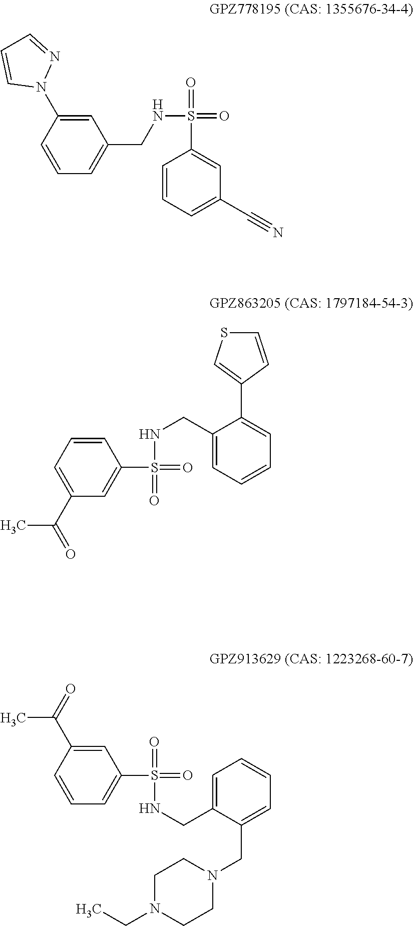

In a particular embodiment, the compound of general formula (I) is selected from the group consisting of:

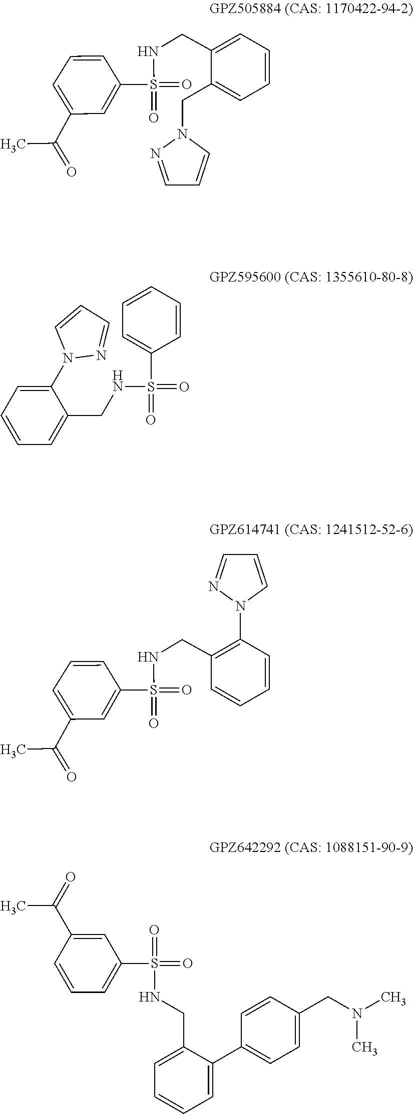

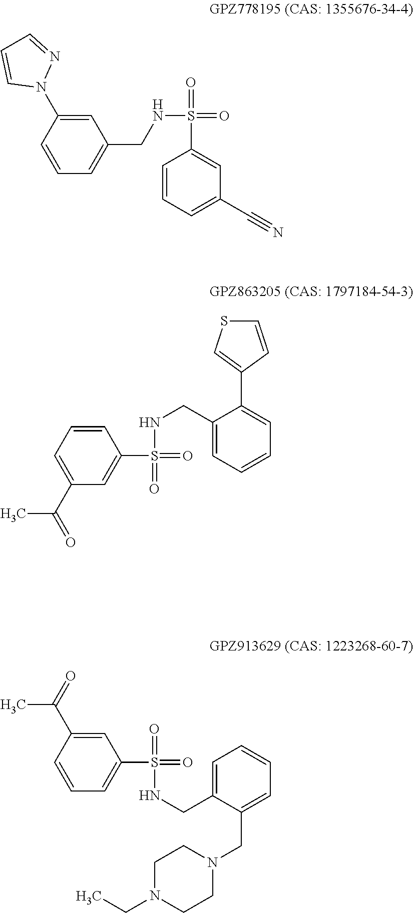

### NGAL Inhibitors of Formula (II)

The present invention also relates to the use of a compound of general formula (II) as a NGAL inhibitor:

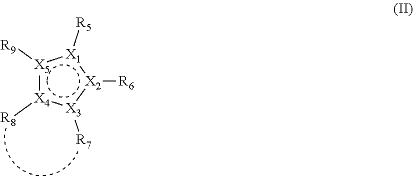

wherein:

X₁ represents a nitrogen atom, or a carbon atom;

X₂ represents a carbon atom, or a CH group;

X₃ represents a nitrogen atom;

X₄ represents an oxygen atom, or a carbon atom;

X₅ represents a carbon atom, or a nitrogen atom;

R₅ represents:
(CH₂)n₄-N(—C₂H₅)(—C₂H₅),
wherein n₄ represents an integer between 0 and 2,
—CH₂—S—R₁₀,
—S—CH₂—R₁₁, or
—C(═O)—N(H)—R₁₂,
wherein R₁₀ R₁₁ and R₁₂ each independently represent an heterocycle of general formula (IV)

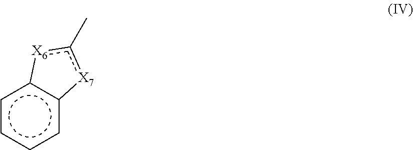

wherein X₆ represents a nitrogen atom, a —NH group, an oxygen atom, or a sulphur atom,
   X₇ represents an oxygen atom or a nitrogen atom,
said heterocycle being optionally substituted by one or more —S(═O)(═O)(—CH₂H₅), —C(═O)Me, halogeno atoms, trifluorometyl groups, cyano groups, or nitro groups;

R₆ represents a phenyl group, a lone pair, an oxygen atom, or an halogeno atom;

R₉ represents a ═NH group, or a lone pair;

R₇ and R₈ represent a lone pair or R₈—X₄—X₃—R₇ optionally form a six membered ring heterocycle, said heterocycle being optionally substituted by one or more methyl groups, preferably by two methyl groups.

In a particular embodiment, a compound wherein:

X₁ represents a carbon atom;

X₂ represents a carbon atom;

X₃ represents a nitrogen atom;

X₄ represents a carbon atom;

X₅ represents a carbon atom;

R₅ represents CH₂—S—R₁₀,
wherein R₁₀ represents an heterocycle of general formula (IV)

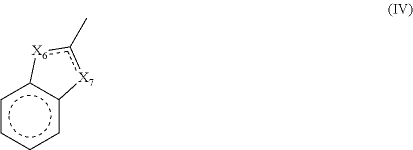

wherein X₆ represents a nitrogen atom, X₇ represents a —NH group,

R₆ represents a lone pair;

R₉ represents a lone pair;

R₈—X₄—X₃—R₇ optionally form a six membered ring heterocycle substituted by one methyl group, is excluded.

In a particular embodiment, a compound wherein:

X₁ represents a nitrogen atom;

X₂ represents a carbon atom;

X₃ represents a nitrogen atom;

X₄ represents an oxygen atom;

X₅ represents a carbon atom;

R₅ represents (CH₂)n₄-N(—C₂H₅)(—C₂H₅) wherein n₄ is equal to 2,

R₆ represents a phenyl group;

R₉ represents a ═NH group;

R₇ and R₈ represent a lone pair, is excluded.

In a particular embodiment, a compound wherein:

X₁ represents a carbon atom;

X₂ represents a carbon atom;

X₃ represents a nitrogen atom;

X₄ represents a carbon atom;

X₅ represents a nitrogen atom;

R₅ represents CH₂—S—R₁₀,
wherein R₁₀ represents an heterocycle of general formula (IV)

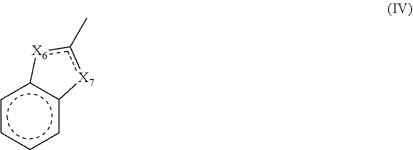

wherein X₆ represents a —NH group, X₇ represents a nitrogen atom,

R₆ represents a lone pair;

R₉ represents a lone pair;

R₈—X₄—X₃—R₇ optionally form a six membered ring heterocycle, is excluded.

In a particular embodiment, the compound of general formula (II) is selected from the group consisting of:

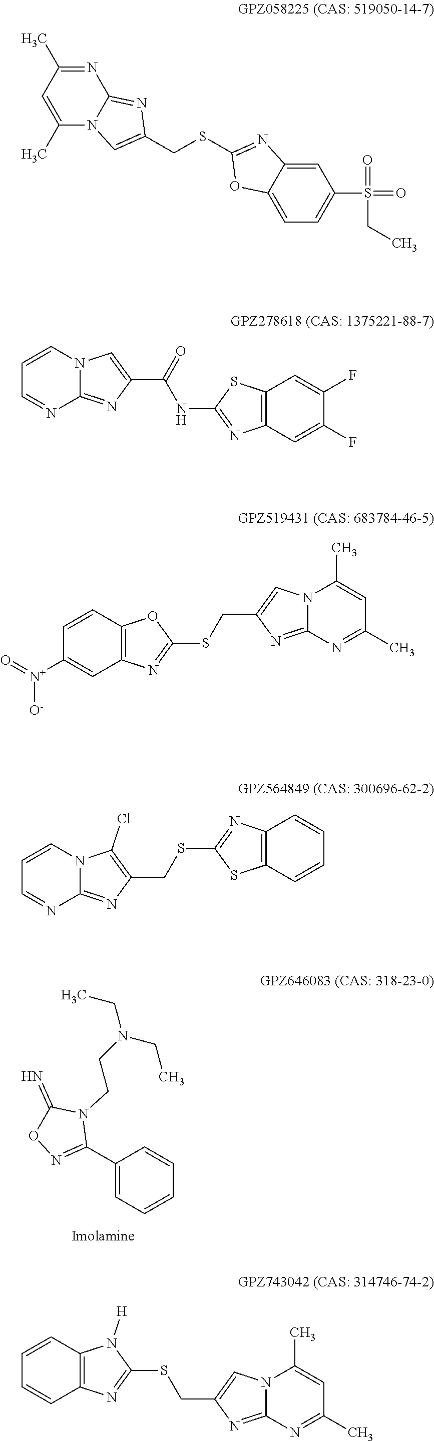

### NGAL Inhibitors of Formula (II)

The present invention also relates to the use of a compound of general formula (III) as a NGAL inhibitor:

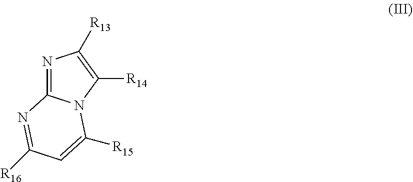

wherein

R₁₃ represents
—CH₂—S—R₁₇,
—S—CH₂—R₁₈, or
—C(═O)—N(H)—R₁₉,
wherein R₁₇, R₁₈ and R₁₉ each independently represent an heterocycle of general formula (V)

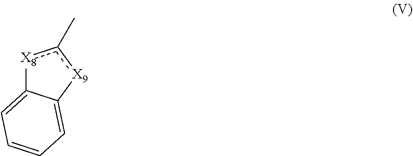

wherein X₈ represents a nitrogen atom, a —NH group, an oxygen atom, or a sulphur atom,
   X₉ represents an oxygen atom or a nitrogen atom,
said heterocycle being optionally substituted by one or more —S(═O)(═O)(—CH₂H₅), halogeno atoms or nitro groups;

R₁₄ represents a lone pair, a hydrogen atom or an halogen atom;

R₁₅ represents a methyl group, a hydrogen atom or a lone pair; and

R₁₆ represents a methyl group, a hydrogen atom or a lone pair.

In a particular embodiment, the inhibitor is selected from the group consisting of:

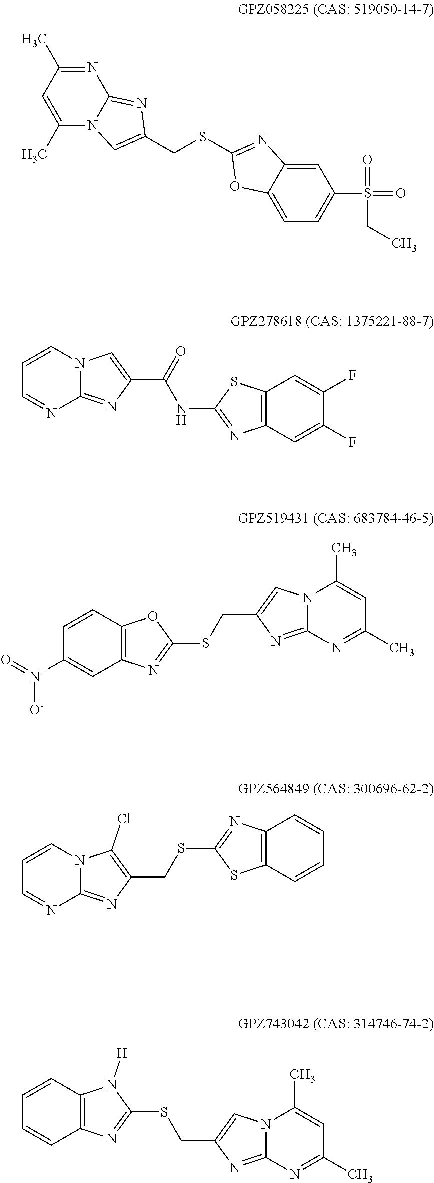 [SMILES: CCS(=O)(=O)c1ccc2oc(SCc3cn4c(C)cc(C)nc4n3)nc2c1 ; O=C(Nc1nc2cc(F)c(F)cc2s1)c1cn2cccnc2n1 ; Cc1cc(C)n2cc(CSc3nc4cc([N+](=O)[O-])ccc4o3)nc2n1 ; Clc1c(CSc2nc3ccccc3s2)nc2ncccn12 ; Cc1cc(C)n2cc(CSc3nc4ccccc4[nH]3)nc2n1]

### Synthesis

### Synthesis of 3-acetyl-N-[2-(1H-pyrazol-1-yl)phenyl]methyl]-benzenesulfonamide (GPZ614741 (CAS 1241512-52-6))

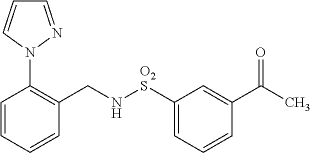 [SMILES: CC(=O)c1cccc(S(=O)(=O)NCc2ccccc2-n2cccn2)c1]

3-acetyl-N-[2-(1H-pyrazol-1-yl)phenyl]methyl]-benzenesulfonamide has been synthetized according to the following scheme.

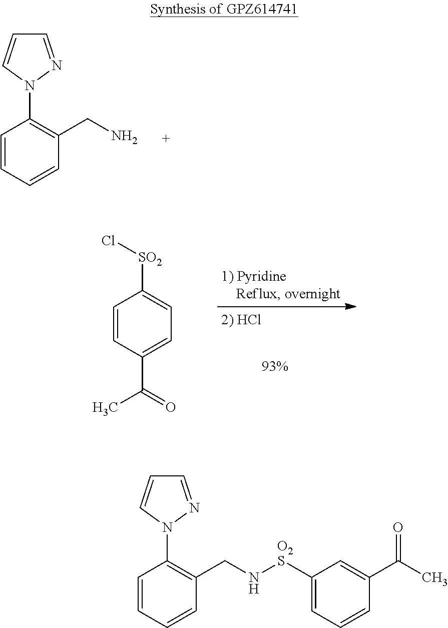 [SMILES: NCc1ccccc1-n1cccn1 ; CC(=O)c1ccc(S(=O)(=O)Cl)cc1 ; CC(=O)c1cccc(S(=O)(=O)NCc2ccccc2-n2cccn2)c1]

To a solution of [2-(1H-pyrazol-1-yl)phenyl]methylamine (3.0 g) in pyridine (20 mL) was slowly added 3-acetylbenzylsulfonyl chloride (4.17 g) at 0° C., and the mixture was stirred overnight at 115° C. After cooling down, the reaction solution was concentrated under reduced pressure. The residue was then dissolved in dichloromethane, washed with HCl 2 N, a saturated aqueous solution of NaHCO3 and saturated brine, then dried over magnesium sulfate, and concentrated under reduced pressure. The residue was finally purified by flash column chromatography (1:1 EtOAc/petroleum ether) to give the title compound as a white solid (5.72 g, 93% yield). 1H NMR (400 MHz, CDCl3) δ 8.27 (t, J=1.6 Hz, 1H), 8.01 (dt, J=1.6 and 8.0 Hz, 1H), 7.96 (dt, J=1.6 and 8.0 Hz, 1H), 7.69 (d, J=1.2 Hz, 1H), 7.63 (d, J=2.4 Hz, 1H), 7.48 (t, J=8.0 Hz, 1H), 7.30-7.26 (m, 2H), 7.18-7.14 (m, 2H), 6.45 (t, J=2.2 Hz, 1H), 4.08 (d, J=6.4 Hz, 2H), 2.55 (s, 3H).

Other compounds are commercialized by Ambinter:

GPZ478519 (CAS: 1171056-55-5)

GPZ505884 (CAS: 1170422-94-2)

GPZ595600 (CAS: 1355610-80-8)

GPZ624624 Sulfaphenazole (CAS: 526-08-9)

GPZ642292 (CAS: 1088151-90-9)

GPZ706277 Acetohexamide (CAS: 968-81-0)

GPZ778195 (CAS: 1355676-34-4)

GPZ863205 (CAS: 1797184-54-3)

GPZ913629 (CAS: 1223268-60-7)

GPZ058225 (CAS: 519050-14-7)

GPZ278618 (CAS: 1375221-88-7)

GPZ519431 (CAS: 683784-46-5)

GPZ564849 (CAS: 300696-62-2)

GPZ646083 Imolamine (CAS: 318-23-0)

GPZ743042 (CAS: 314746-74-2)

### Method

The present invention also relates to a method for inhibiting NGAL activity by using the compound as previously defined in the presence of a NGAL protein.

Preferably, the compound is a compound of formula (I) as previously defined.

Preferably, the compound is a compound of formula (II) as previously defined.

Preferably, the compound is a compound of formula (III) as previously defined.

According to one embodiment, the present invention relates to a therapeutic use of the method as defined above.

In another embodiment, the present invention relates to non-therapeutic use of the method as defined above.

### Therapeutic Use

It is known from the prior art that NGAL protein is involved in several diseases.

The present inventors have found and demonstrated the inhibitory activity of compounds as previously defined towards NGAL protein. Accordingly, it will be acknowledged that the compounds as previously defined, which are NGAL inhibitors, can be used for treating NGAL induced diseases.

The present inventors have found that the compounds as defined in the present invention can be used as therapeutic agents.

Thus, the present invention relates to the compound as previously defined for use in a therapeutic method for inhibiting the NGAL activity.

Preferably, the compound is a compound of formula (I) as previously defined.

Preferably, the compound is a compound of formula (II) as previously defined.

Preferably, the compound is a compound of formula (III) as previously defined.

Thus, the present invention relates to the compound as previously defined for its use as a medicament.

Preferably, the compound is a compound of formula (I) as previously defined.

Preferably, the compound is a compound of formula (II) as previously defined.

Preferably, the compound is a compound of formula (III) as previously defined.

The present invention also provides a pharmaceutical composition comprising, as active principle, the compound as previously defined and a pharmaceutically acceptable excipient.

Preferably, the compound is a compound of formula (I) as previously defined.

Preferably, the compound is a compound of formula (II) as previously defined.

Preferably, the compound is a compound of formula (III) as previously defined.

The term “pharmaceutical composition” in the present invention refers to any composition comprising compound of formula (I), the compound of formula (II) or the compound of formula (III) as previously defined and at least one pharmaceutically acceptable excipient. By the term “pharmaceutically acceptable excipient” herein, it is understood a carrier medium which does not interfere with the effectiveness of the biological activity of the active ingredient(s) and which is not excessively toxic to the host at the concentration at which it is administered. Said excipients are selected, depending on the pharmaceutical form and the desired method of administration, from the usual excipients known by a person skilled in the art.

In particular, the compound as previously defined can be used for treating NGAL induced diseases.

Preferably, the compound is a compound of formula (I) as previously defined.

Preferably, the compound is a compound of formula (II) as previously defined.

Preferably, the compound is a compound of formula (III) as previously defined.

Thus, the present invention also relates to the compound as previously defined for the manufacture of a medicament for the prevention or treatment of a NGAL induced disease.

Preferably, the compound is a compound of formula (I) as previously defined.

Preferably, the compound is a compound of formula (II) as previously defined.

Preferably, the compound is a compound of formula (III) as previously defined.

By “NGAL induced diseases” it is understood diseases mediated by the NGAL protein. It is well-known that diseases mediated by the NGAL protein refer to any disease where the NGAL protein plays a role.

As used herein “NGAL induced diseases” refers to a specific form of disease wherein the NGAL contributes to the development of the disease and thus refers to a particular sub-type of diseases.

Typically, to know if the disease to be treated or prevented is a NGAL induced disease, the expression and/or activity of NGAL protein can be measured using ELISA, western-blot or any quantitative protein or gene expression methods. A particular increase of NGAL expression/activity in tissues, cells or body fluids will indicate that NGAL contribute to the development of a particular sub-type of diseases.

According to the present invention, by treating NGAL induced diseases it is also understood preventing NGAL induced diseases. Indeed, by treating a disease associated with the presence of NGAL protein, it is possible to prevent a disease or a trouble linked to the presence of NGAL protein.

Thus, in particular, the compound as previously defined can be used for preventing NGAL induced diseases.

Preferably, the compound is a compound of formula (I) as previously defined.

Preferably, the compound is a compound of formula (II) as previously defined.

Preferably, the compound is a compound of formula (III) as previously defined.

Another object of the present invention relates to a method of treating a NGAL induced disease, comprising administering to a patient in need thereof, an effective amount of a compound as previously defined, or of a pharmaceutical composition comprising said compound.

Preferably, the compound is a compound of formula (I) as previously defined.

Preferably, the compound is a compound of formula (II) as previously defined.

Preferably, the compound is a compound of formula (III) as previously defined.

Another object of the present invention relates to a method of preventing a NGAL induced disease, comprising administering to a patient in need thereof, an effective amount of a compound as previously defined, or of a pharmaceutical composition comprising said compound.

Preferably, the compound is a compound of formula (I) as previously defined.

Preferably, the compound is a compound of formula (II) as previously defined.

Preferably, the compound is a compound of formula (III) as previously defined.

By “an effective amount” is meant a sufficient amount of the compound of formula (I), the compound of formula (II) or the compound of formula (III) to treat or to prevent the NGAL induced disease.

Suitable dosage ranges depend upon numerous factors such as the severity of the disease to be treated, the age and relative health of the subject, the route and the form of administration.

According to the present invention, the compound of formula (I), the compound of formula (II) or the compound of formula (III) may be administered alone or in combination with other drugs well known by a person skilled in the art.

Typically, the patient may be a human or another mammal (e.g., primate, mouse, rat, rabbit, dog, cat, horse, cow, pig, camel, and the like). Preferably, the patient is a human.

In a particular embodiment, NGAL induced diseases are cardiovascular diseases.

The role of NGAL in cardiovascular diseases or troubles has been studied in:

a) Heart failure, and in particular hypertrophic heart failure:
Lipocalin-2 induces NLRP3 inflammasome activation via HMGB1 induced TLR4 signaling in heart tissue of mice under pressure overload challenge, Song E, Jahng J W, Chong L P, Sung H K, Han M, Luo C, Wu D, Boo S, Hinz B, Cooper M A, Robertson A A, Berger T, Mak T W, George I, Schulze P C, Wang Y, Xu A, Sweeney G. Am J Transl Res. 2017 Jun. 15; 9(6):2723-2735. eCollection 2017.

b) Abdominal aortic aneurysm:
Lipocalin-2 deficiency or blockade protects against aortic abdominal aneurysm development in mice, Tarin C, Fernandez-Garcia C E, Burillo E, Pastor-Vargas C, Llamas-Granda P, Castejón B, Ramos-Mozo P, Torres-Fonseca M M, Berger T, Mak T W, Egido J, Blanco-Colio L M, Martin-Ventura J L. Cardiovasc Res. 2016 Aug. 1; 111(3):262-73.

c) Ischemia reperfusion in transplanted heart, cardiac ischemia reperfusion, besides transplantation:
Lipocalin-2 regulates the inflammatory response during ischemia and reperfusion of the transplanted heart, Aigner F, Maier H T, Schwelberger H G, Wallnofer E A, Amberger A, Obrist P, Berger T, Mak T W, Maglione M, Margreiter R, Schneeberger S, Troppmair J. Am J Transplant. 2007 April; 7(4):779-88,

d) Atherosclerosis:
Lipocalin-2 contributes to experimental atherosclerosis in a stage-dependent manner, Amersfoort J, Schaftenaar F H, Douna H, van Santbrink P J, Kroner M J, van Puijvelde G H M, Quax P H A, Kuiper J, Bot I. Atherosclerosis. 2018 August; 275:214-224,
More than a simple biomarker: the role of NGAL in cardiovascular and renal diseases, Buonafine M, Martinez-Martinez E, Jaisser F, Clin Sci (Lond). 2018 May 8; 132(9):909-923.

As disclosed in WO 2012/072820 A1, inhibitors of NGAL gene expression or NGAL antagonists may be used in the prevention or the treatment of heart failure.

The term “heart failure” (HF) as used herein embraces congestive heart failure and/or chronic heart failure. Functional classification of heart failure is generally done by the New York Heart Association Functional Classification (Criteria Committee, New York Heart Association. Diseases of the heart and blood vessels. Nomenclature and criteria for diagnosis, 6th ed. Boston: Little, Brown and co, 1964; 114). This classification stages the severity of heart failure into 4 classes (I-IV). The classes (I-IV) are:
Class I: no limitation is experienced in any activities; there are no symptoms from ordinary activities,
Class II: slight, mild limitation of activity; the patient is comfortable at rest or with mild exertion, and
Class III: marked limitation of any activity; the patient is comfortable only at rest. Class IV: any physical activity brings on discomfort and symptoms occur at rest.

WO 2013/156867 A1 discloses that inhibitors of NGAL activity or gene expression may be used in the prevention or in the treatment of hypertension including arterial hypertension, venous hypertension and pulmonary hypertension.

WO 2014/049152 A1 relates to an in inhibitor of NGAL activity or gene expression for use in a method for treating or preventing cardiovascular fibrosis.

Thus, the present invention relates to the compound as previously defined for use in the treatment of heart failure, cardiac infarct, hypertension, cardiovascular fibrosis, atherosclerosis, cardiac ischemia-reperfusion injury, abdominal aortic aneurysm in a patient.

Preferably, the compound is a compound of formula (I) as previously defined.

Preferably, the compound is a compound of formula (II) as previously defined.

Preferably, the compound is a compound of formula (III) as previously defined.

Thus, the present invention also relates to the compound as previously defined for use in the prevention of heart failure, cardiac infarct, hypertension, cardiovascular fibrosis, atherosclerosis, cardiac ischemia-reperfusion injury, abdominal aortic aneurysm in a patient.

Preferably, the compound is a compound of formula (I) as previously defined.

Preferably, the compound is a compound of formula (II) as previously defined.

Preferably, the compound is a compound of formula (III) as previously defined.

According to the present invention, hypertension includes arterial hypertension, venous hypertension and pulmonary hypertension.

The term “hypertension” may also refer to arterial hypertension associated with chronic renal failure and/or to arterial hypertension and/or salt induced hypertension.

Thus, the present invention relates to the compound as previously defined for use in the prevention of heart failure in a patient.

Preferably, the compound is a compound of formula (I) as previously defined.

Preferably, the compound is a compound of formula (II) as previously defined.

Preferably, the compound is a compound of formula (III) as previously defined.

In a particular embodiment, the NGAL induced diseases are renal diseases.

The role of NGAL in renal diseases or troubles has been studied in:
Lipocalin 2 is essential for chronic kidney disease progression in mice and humans, Viau A, El Karoui K, Laouari D, Burtin M, Nguyen C, Mori K, Pillebout E, Berger T, Mak T W, Knebelmann B, Friedlander G, Barasch J, Terzi F. J Clin Invest. 2010 November; 120(11):4065-76,
Lipocalin-2 protects against renal ischemia reperfusion injury in mice through autophagy activation mediated by HIF1α and NF-κb crosstalk, Qiu S, Chen X, Pang Y, Zhang Z. Biomed Pharmacother. 2018 Sep. 13; 108:244-253,
Lipocalin-2 derived from adipose tissue mediates aldosterone-induced renal injury, Sun W Y, Bai B, Luo C, Yang K, Li D, Wu D, Félétou M, Villeneuve N, Zhou Y, Yang J, Xu A, Vanhoutte P M, Wang Y. JCI Insight. 2018 Sep. 6; 3(17),
Neutrophil gelatinase-associated lipocalin is instrumental in the pathogenesis of antibody-mediated nephritis in mice, Pawar R D, Pitashny M, Gindea S, Tieng A T, Levine B, Goilav B, Campbell S R, Xia Y, Qing X, Thomas D B, Herlitz L, Berger T, Mak T W, Putterman C. Arthritis Rheum. 2012 May; 64(5):1620-31,
More than a simple biomarker: the role of NGAL in cardiovascular and renal diseases, Buonafine M, Martinez-Martinez E, Jaisser F, Clin Sci (Lond). 2018 May 8; 132(9):909-923.

Typically, renal diseases are chronic kidney diseases, renal ischemia-reperfusion injury, aldosterone-induced renal injury, renal fibrosis, and antibody-mediated nephritis.

Thus, the present invention relates to the compound as previously defined for use in the treatment of renal diseases including chronic kidney diseases, renal ischemia-reperfusion injury, aldosterone-induced renal injury, renal fibrosis and antibody-mediated nephritis.

Preferably, the compound is a compound of formula (I) as previously defined.

Preferably, the compound is a compound of formula (II) as previously defined.

Preferably, the compound is a compound of formula (III) as previously defined.

Thus, the present invention relates to the compound as previously defined for use in the prevention of renal diseases including chronic kidney diseases, renal ischemia-reperfusion injury, aldosterone-induced renal injury, renal fibrosis and antibody-mediated nephritis. According to the present invention, renal injury includes inflammation and renal dysfunction.

According to the present invention, “renal fibrosis” may refer to renal interstitial fibrosis associated with chronic renal failure.

Preferably, the compound is a compound of formula (I) as previously defined.

Preferably, the compound is a compound of formula (II) as previously defined.

Preferably, the compound is a compound of formula (III) as previously defined.

In a particular embodiment, NGAL induced diseases are associated to metabolism and obesity.

The role of NGAL in metabolism and obesity has been studied in:
Lipocalin-2 deficiency prevents endothelial dysfunction associated with dietary obesity: role of cytochrome P450 2C inhibition, Liu J T, Song E, Xu A, Berger T, Mak T W, Tse H F, Law I K, Huang B, Liang Y, Vanhoutte P M, Wang Y. Br J Pharmacol. 2012 January; 165(2):520-31.
Lipocalin-2 deficiency attenuates insulin resistance associated with aging and obesity, Law I K, Xu A, Lam K S, Berger T, Mak T W, Vanhoutte P M, Liu J T, Sweeney G, Zhou M, Yang B, Wang Y Diabetes. 2010 April; 59(4):872-82,
Obesity-promoting and anti-thermogenic effects of neutrophil gelatinase-associated lipocalin in mice, Ishii A, Katsuura G, Imamaki H, Kimura H, Mori K P, Kuwabara T, Kasahara M, Yokoi H, Ohinata K, Kawanishi T, Tsuchida J, Nakamoto Y, Nakao K, Yanagita M, Mukoyama M, Mori K. Sci Rep. 2017 Nov. 14; 7(1):15501,
Deamidated lipocalin-2 induces endothelial dysfunction and hypertension in dietary obese mice, Song E, Fan P, Huang B, Deng H B, Cheung B M, F616tou M, Vilaine J P, Villeneuve N, Xu A, Vanhoutte P M, Wang Y. J Am Heart Assoc. 2014 Apr. 10; 3(2):e000837,
Evidence for the regulatory role of lipocalin 2 in high-fat diet-induced adipose tissue remodeling in male mice, Guo H, Bazuine M, Jin D, Huang M M, Cushman S W, Chen X. Endocrinology. 2013 October; 154(10):3525-38.

Thus, the present invention relates to a compound as previously defined for use in the treatment of obesity and diabetes, insulin resistance associated to aging, endothelial dysfunction associated with dietary obesity.

Preferably, the compound is a compound of formula (I) as previously defined.

Preferably, the compound is a compound of formula (II) as previously defined.

Preferably, the compound is a compound of formula (III) as previously defined.

Thus, the present invention relates to a compound as previously defined for use in the prevention of obesity and diabetes, insulin resistance associated to aging, endothelial dysfunction associated with dietary obesity.

Preferably, the compound is a compound of formula (I) as previously defined.

Preferably, the compound is a compound of formula (II) as previously defined.

Preferably, the compound is a compound of formula (III) as previously defined.

In a particular embodiment, NGAL induced diseases are cancer.

The role of NGAL in cancer has been studied in:
Disruption of the Lcn2 gene in mice suppresses primary mammary tumor formation but does not decrease lung metastasis, Berger T, Cheung C C, Elia A J, Mak T W. Proc Natl Acad Sci USA. 2010 Feb. 16; 107(7):2995-3000,
Tumour stroma-derived lipocalin-2 promotes breast cancer metastasis, Ören B, Urosevic J, Mertens C, Mora J, Guiu M, Gomis R R, Weigert A, Schmid T, Grein S, Brune B, Jung M. J Pathol. 2016 July; 239(3):274-85,
Knockdown of lipocalin-2 suppresses the growth and invasion of prostate cancer cells, Tung M C, Hsieh S C, Yang S F, Cheng C W, Tsai R T, Wang S C, Huang M H, Hsieh Y H. Prostate. 2013 September; 73(12):1281-90,
Requirement of lipocalin 2 for hematopoietic and solid tumor malignancies, Leng X, Ding T, Arlinghaus R. Adv Enzyme Regul. 2009; 49(1):142-6. doi: 10.1016/j,
Lipocalin-2 Promotes Pancreatic Ductal Adenocarcinoma by Regulating Inflammation in the Tumor Microenvironment Sobeyda B. Gomez-Chou, Agnieszka Katarzyna Swidnicka-Siergiejko, Niharika Badi, Myrriah Chavez-Tomar, Gregory B. Lesinski, Tanios Bekaii-Saab, Matthew R. Farren, Thomas A. Mace, Carl Schmidt, Yan Liu, Defeng Deng, Rosa F. Hwang, Liran Zhou, Todd Moore, Deyali Chatterjee, Huamin Wang, Xiaohong Leng, Ralph B. Arlinghaus, Craig D. Logsdon and Zobeida Cruz-Monserrate. Cancer Res; 77(10); 2647-60.
Lipocalin 2 in cancer: when good immunity goes bad, Rodvold, Mahadevan N R, Zanetti M, Cancer Lett. 2012 Mar. 28; 316(2):132-8.

Typically, cancer includes lung cancer, breast cancer, prostate cancer, pancreatic cancer, and chronic myeloid leukemia.

Thus, the present invention relates to the compound as previously defined for use in the treatment of cancer including lung cancer, breast cancer, prostate cancer, pancreatic cancer and chronic myeloid leukemia.

Preferably, the compound is a compound of formula (I) as previously defined.

Preferably, the compound is a compound of formula (II) as previously defined.

Preferably, the compound is a compound of formula (III) as previously defined.

Thus, the present invention relates to the compound as previously defined for use in the prevention of cancer including lung cancer, breast cancer, prostate cancer, pancreatic cancer and chronic myeloid leukemia.

Preferably, the compound is a compound of formula (I) as previously defined.

Preferably, the compound is a compound of formula (II) as previously defined.

Preferably, the compound is a compound of formula (III) as previously defined.

In a particular embodiment, NGAL induced diseases are inflammatory diseases.

The role of NGAL in inflammatory diseases has been studied in:
Increased Lipocalin-2 in the retinal pigment epithelium of Cryba1 cKO mice is associated with a chronic inflammatory response, Valapala M, Edwards M, Hose S, Grebe R, Bhutto I A, Cano M, Berger T, Mak T W, Wawrousek E, Handa J T, Lutty G A, Samuel Zigler J Jr, Sinha D. Aging Cell. 2014 December; 13(6):1091-4,
Lipocalin-2 protein deficiency ameliorates experimental autoimmune encephalomyelitis: the pathogenic role of lipocalin-2 in the central nervous system and peripheral lymphoid tissues, Nam Y, Kim J H, Seo M, Kim J H, Jin M, Jeon S, Seo J W, Lee W H, Bing S J, Jee Y, Lee W K, Park D H, Kook H, Suk K. J Biol Chem. 2014 Jun. 13; 289(24):16773-89,
A possible contribution of lipocalin-2 to the development of dermal fibrosis, pulmonary vascular involvement and renal dysfunction in systemic sclerosis, Takahashi T, Asano Y, Noda S, Aozasa N, Akamata K, Taniguchi T, Ichimura Y, Toyama T, Sumida H, Kuwano Y, Tada Y, Sugaya M, Kadono T, Sato S. Br J Dermatol. 2015 September; 173(3):681-9,
Lipocalin 2 is a novel immune mediator of experimental autoimmune encephalomyelitis pathogenesis and is modulated in multiple sclerosis, Berard J L, Zarruk J G, Arbour N, Prat A, Yong V W, Jacques F H, Akira S, David S. Glia. 2012 July; 60(7):1145-59,
Lipocalin-2: A Master Mediator of Intestinal and Metabolic Inflammation, Moschen, Adolph, Gerner, Wieser, Tilg H, Trends Endocrinol Metab. 2017 May; 28(5):388-397.

Thus, the present invention relates to the compound as previously defined for use in the treatment of inflammatory diseases including intestinal inflammation, retinal diseases, systemic sclerosis, and encephalomyelitis.

Preferably, the compound is a compound of formula (I) as previously defined.

Preferably, the compound is a compound of formula (II) as previously defined.

Preferably, the compound is a compound of formula (III) as previously defined.

Thus, the present invention relates to the compound as previously defined for use in the prevention of inflammatory diseases including intestinal inflammation, retinal diseases, systemic sclerosis, and encephalomyelitis.

Preferably, the compound is a compound of formula (I) as previously defined.

Preferably, the compound is a compound of formula (II) as previously defined.

Preferably, the compound is a compound of formula (III) as previously defined.

In a particular embodiment, NGAL induced diseases are linked to neuroprotection troubles.

The role of NGAL in neuroprotection troubles has been studied in:
Astrocyte-derived lipocalin-2 mediates hippocampal damage and cognitive deficits in experimental models of vascular dementia, Kim J H, Ko P W, Lee H W, Jeong J Y, Lee M G, Kim J H, Lee W H, Yu R, Oh W J, Suk K. Glia. 2017 September; 65(9):1471-1490,
Role of Lipocalin-2 in Thrombin-Induced Brain Injury, Mao S, Xi G, Keep R F, Hua Y. Stroke. 2016 April; 47(4):1078-84,
Lack of NG2 exacerbates neurological outcome and modulates glial responses after traumatic brain injury, Huang C, Sakry D, Menzel L, Dangel L, Sebastiani A, Krämer T, Karram K, Engelhard K, Trotter J, Schafer M K. Glia. 2016 April; 64(4):507-23,
Lipocalin-2 is a pathogenic determinant and biomarker of neuropsychiatric lupus, Mike E V, Makinde H M, Gulinello M, Vanarsa K, Herlitz L, Gadhvi G, Winter D R, Mohan C, Hanly J G, Mok C C, Cuda C M, Putterman C. J Autoimmun. 2018 Aug. 30. pii: S0896-8411(18)30387-1,
Pathogenic Upregulation of Glial Lipocalin-2 in the Parkinsonian Dopaminergic System, Kim B W, Jeong K H, Kim J H, Jin M, Kim J H, Lee M G, Choi D K, Won S Y, McLean C, Jeon M T, Lee H W, Kim S R, Suk K. J Neurosci. 2016 May 18; 36(20):5608-22,
Lipocalin-2 deficiency attenuates neuroinflammation and brain injury after transient middle cerebral artery occlusion in mice, Jin M, Kim J H, Jang E, Lee Y M, Soo Han H, Woo D K, Park D H, Kook H, Suk K. J Cereb Blood Flow Metab. 2014 August; 34(8):1306-14,
Role of lipocalin-2-chemokine axis in the development of neuropathic pain following peripheral nerve injury, Jeon S, Jha M K, Ock J, Seo J, Jin M, Cho H, Lee W H, Suk K. J Biol Chem. 2013 Aug. 16; 288(33):24116-27,
Lipocalin-2 as a therapeutic target for brain injury: An astrocentric perspective, Suk K. Prog Neurobiol. 2016 September; 144:158-72,
Pathological Involvement of Astrocyte-Derived Lipocalin-2 in the Demyelinating Optic Neuritis, Chun B Y, Kim J H, Nam Y, Huh M I, Han S, Suk K. Invest Ophthalmol Vis Sci. 2015 June; 56(6):3691-8,
Role of lipocalin-2 in brain injury after intracerebral hemorrhage, Ni W, Zheng M, Xi G, Keep R F, Hua Y. J Cereb Blood Flow Metab. 2015 September; 35(9):1454-61.

Thus, the present invention relates to the compound as previously defined for use in the treatment of Parkinson, Alzheimer, vascular dementia, stoke, transient ischemic event, traumatic brain injury, neuropathic pain, and optic nerve neuritis.

Preferably, the compound is a compound of formula (I) as previously defined.

Preferably, the compound is a compound of formula (II) as previously defined.

Preferably, the compound is a compound of formula (III) as previously defined.

Thus, the present invention relates to the compound as previously defined for use in the prevention of Parkinson, Alzheimer, vascular dementia, stoke, transient ischemic event, traumatic brain injury, neuropathic pain, and optic nerve neuritis.

Preferably, the compound is a compound of formula (I) as previously defined.

Preferably, the compound is a compound of formula (II) as previously defined.

Preferably, the compound is a compound of formula (III) as previously defined.

In a particular embodiment, NGAL induced diseases are liver diseases.

The role of NGAL in liver diseases has been studied in:
The Detrimental Role Played by Lipocalin-2 in Alcoholic Fatty Liver in Mice, Cai Y, Jogasuria A, Yin H, Xu M J, Hu X, Wang J, Kim C, Wu J, Lee K, Gao B, You M. Am J Pathol. 2016 September; 186(9):2417-28. doi: 10.1016/j,

Thus, the present invention relates to the compound as previously defined for use in the treatment of alcoholic fatty liver diseases.

Preferably, the compound is a compound of formula (I) as previously defined.

Preferably, the compound is a compound of formula (II) as previously defined.

Preferably, the compound is a compound of formula (III) as previously defined.

Thus, the present invention relates to the compound as previously defined for use in the prevention of alcoholic fatty liver diseases.

Preferably, the compound is a compound of formula (I) as previously defined.

Preferably, the compound is a compound of formula (II) as previously defined.

Preferably, the compound is a compound of formula (III) as previously defined.

In particular, the compound as previously defined can be used for the treatment of a wound and in particular a chronic wound. Also, the compound as previously defined can be used for the treatment of a delayed wound closure.

Preferably, the compound is a compound of formula (I) as previously defined.

Preferably, the compound is a compound of formula (II) as previously defined.

Preferably, the compound is a compound of formula (III) as previously defined.

In particular, the compound as previously defined can be used for the prevention of a wound and in particular a chronic wound. Also, the compound as previously defined can be used for the prevention of a delayed wound closure.

Preferably, the compound is a compound of formula (I) as previously defined.

Preferably, the compound is a compound of formula (II) as previously defined.

Preferably, the compound is a compound of formula (III) as previously defined.

By “chronic wound” or “delayed wound closure” it is understood a wound that does not heal in an orderly set of stages and in a predictable amount of time.

Typically, wounds that do not heal within three months are considered chronic. Examples of chronic wounds include, but are not limited to, venous stasis ulcers, diabetic ulcers such as diabetic foot ulcers, and the like. Chronic, wounds may also include those relating to trauma or repeated trauma, thermal injury (e.g., burns) and radiation damage. In some embodiments, the treatment of chronic wound in a subject suffering from sickle-cell disease is also encompassed. In some embodiments, the treatment of chronic wound in elderly persons is also encompassed.

Another object of the present invention relates to a method of treating a wound, in particular a chronic wound, and a delayed wound closure, comprising applying to the skin subject an effective amount of a compound as previously defined, or a pharmaceutical composition comprising said compound.

Preferably, the compound is a compound of formula (I) as previously defined.

Preferably, the compound is a compound of formula (II) as previously defined.

Preferably, the compound is a compound of formula (III) as previously defined.

Another object of the present invention relates to a method of preventing a wound, in particular a chronic wound, and a delayed wound closure, comprising applying to the skin subject an effective amount of a compound as previously defined, or a pharmaceutical composition comprising said compound.

Preferably, the compound is a compound of formula (I) as previously defined.

Preferably, the compound is a compound of formula (II) as previously defined.

Preferably, the compound is a compound of formula (III) as previously defined.

By “an effective amount” is meant a sufficient amount of the compound of formula (I), the compound of formula (II) or the compound of formula (III) to treat or to prevent the wound, in particular the chronic wound, and the delayed wound closure.

Suitable dosage ranges depend upon numerous factors such as the severity of the wound to be treated, the age and relative health of the subject, and the form of the pharmaceutical composition.

According to the present invention, the compound of formula (I), the compound of formula (II) or the compound of formula (III) may be applied on the skin of the subject alone or in combination with other drugs well known by a person skilled in the art.

Typically, the subject may be a human or another mammal (e.g., primate, mouse, rat, rabbit, dog, cat, horse, cow, pig, camel, and the like). Preferably, the patient is a human.

Typically, the subject may suffer of a venous stasis ulcer, diabetic ulcer such as diabetic foot ulcer, trauma or repeated trauma, thermal injury such as burn, radiation damage and sickle-cell disease.

Although the exemplary embodiments of the present invention have been disclosed for illustrative purposes, a person skilled in the art will appreciate that various modifications, additions and substitutions are possible, without departing from the scope and spirit of the invention as disclosed in the accompanying claims.

The present invention will now be illustrated using the following examples and figures, which are given by way of illustration, and are in no way limiting.

### BRIEF DESCRIPTION OF DRAWINGS

FIG. 1: Efficacy of GPZ614741 to inhibit NGAL induced secretion of IL6 (panel A) or Col1 (panel B), NGAL-induced increased cellular content of Col1, col III, fibronectin, Gal3, CT1 or OPN (panel C) or aldo-induced expression of Col1, Col3 and IL6 (panel D).

FIG. 2: Efficacy of GPZ058225 to inhibit NGAL-induced secretion of IL6 (panel A) or Col1 (panel B), NGAL-induced increased cellular content of Col1, col III, fibronectin, Gal3, CT1 or OPN (panel C) or aldo-induced expression of Col1, Col3 and IL6 (panel D).

FIG. 3: Inhibitory effects of 4 compounds GP3 (GPZ706277) (panel A), GP4 (GPZ646083) (panel B), GP6 (GPZ624624) (panel C) on NGAL-induced proliferation of human cardiac fibroblast cells.

FIG. 4: Efficacy of GP3 (GPZ706277) to inhibit NGAL-induced secretion of IL6 (panel A) or Col1 (panel B) and aldo-induced secretion of IL6 (panel C) or Col1 (panel D).

FIG. 5: Efficacy of GP4 (GPZ646083) to inhibit NGAL-induced secretion of IL6 (panel A) or Col1 (panel B) and aldo-induced secretion of IL6 (panel C) or Col1 (panel D).

FIG. 6: Efficacy of GP6 (GPZ624624) to inhibit NGAL-induced secretion of IL6 (panel A) or Col1 (panel B) and aldo-induced secretion of IL6 (panel C) or Col1 (panel D).

FIG. 7: Beneficial effect of GPZ614741 administration on functional cardiac parameters upon myocardial infarct.

FIG. 8: Beneficial effect of GPZ614741 administration on functional cardiac parameters upon myocardial infarct.

FIG. 9: Beneficial effect of GP1 (GPZ614741) administration on cardiac fibrosis upon myocardial infarct.

FIG. 10: Beneficial effect of GP1 (GPZ614741) administration on cardiac profibrotic cardiac gene expression upon myocardial infarct.

FIG. 11: Beneficial effect of GP1 (GPZ614741) administration on cardiac IL6 gene expression upon myocardial infarct.

FIG. 12: Beneficial effect of GP1 (GPZ614741) administration on cardiac inflammatory gene expression upon myocardial infarct.

FIG. 13: Beneficial effect of GP1 (GPZ614741) administration on blood pressure increase upon chronic kidney disease.

FIG. 14: Beneficial effect of GP1 (GPZ614741) administration on renal fibrosis upon chronic kidney disease.

FIG. 15: Beneficial effect of GP1 (GPZ614741) administration on renal profibrotic (but not inflammatory) gene expression upon chronic kidney disease.

FIG. 16: Efficacy of GP1 (GPZ614741) to inhibit NGAL induced expression of collagen1, fibronectin and aSMA (panel A)(panel B) and NGAL-induced expression of IL6 and MCP1 (panel B).

FIG. 17: Efficacy of GP1 (GPZ614741) to prevent the increase of blood pressure induced by the combination of L-NAME and High salt Diet.

### EXAMPLES

### Example 1: In Silico Screening of Potential Inhibitors of NGAL

Prior to the virtual screening on NGAL, a database of compounds for virtual screening was prepared and the feasibility of in silico screening models for NGAL inhibitors was studied.

(i) Database of Compounds for Virtual Screening

More than 100 suppliers of chemical molecules were contacted for their catalogues to enrich the www.ambinter.com database. The aim is to have access to molecules, as much as possible, for future virtual and real screening. These chemical catalogues are usually in different formats which are not directly exploitable. A procedure to “normalize” the structures to be in the database was developed: discard radioactive, metallic, metalloid products, reactive products, pan-assay interference compounds, separate salts from main compounds, normalize the informatic representation of chemical structures (aromatization, ionic states etc.), calculate the 3D structure of each compounds. For natural compounds there can be specific problems such as fused rings or missing chirality information. In that case, the most probable configurations according to their internal energies was calculated. Unity molecular fingerprints (which can be seen as bar-codes that identify molecules) were calculated for the molecules for subsequent diversity or similarity searches. Subsets of the database consisting in natural products, drug-like molecules, lead-like molecules and molecules interfering with protein-protein interaction were also built.

(ii) In Silico Screening Models for NGAL Inhibitors

There are 2 possibilities to build protein models for virtual screening. If the structure of co-crystallised ligand-protein complex exists then the co-crystallised ligand can be used to calibrate the virtual screening software (the in silico prediction of how the ligand binds to the protein must fit with the X-ray data). If no structure is available, the target structure can be modeled by homology (for example find a homologue with a known 3D structure and use it as a template). The search of relevant homologue is performed with Fugue and the building of the molecule structure with Orchestrar within Sybyl package (Tripos, Mo., USA).

NGAL is an iron transporter and a signaling protein. Several crystal structures of NGAL with siderophores are published in the Protein Data Bank (e.g. PDB accession codes: 1DFV, 1NGL, etc.). By examining these 3D structures with or without siderophores, it is clear that the general 3D structure of the protein does not change. Therefore targeting the “main” binding site of siderophores may not exert significant effects though this cannot be rule out. The biological activity of NGAL in the context of MIF may be related to its interactions with a receptor. As NGAL receptor could not be modelled (no crystallographic data and no reliable homologue with a known 3D structure), the chosen strategy was to block the interaction of NGAL with its receptor by identifying putative protein-protein interaction (PPI) disruptors. Based on the 3D structure of NGAL, potential “hot spots” (i.e. residues important for PPI) on the protein surface were identified.

### Results

2 peripheral sites that may be PPI zones and be druggable were identified. Virtual screening was used to select potential disruptors of NGAL-NGAL receptor interaction. It consists in simulating the binding energy of a small molecule with NGAL binding sites. Since there are significant differences between NMR and X-ray 3D structures of NGAL (RMSD=4.6 Å calculated according to the backbone of 1DFV and 1NGL), it was necessary to use these 2 structures as templates for modelling NGAL.

### Example 2: In Vivo and In Vitro Studies

Cell Culture Human cardiac fibroblasts (primary culture) were obtained from Promocell and maintained in medium (Fibroblast Media 3). Cells were cultured according to the manufacturer's instructions and used between passages 5 and 7. Cells were stimulated with aldosterone (10-8 M, Sigma) or recombinant hNGAL (500 ng/mL, R&D Systems) for 24 h for protein analysis. Mouse kidney fibroblasts (MKF), were isolated from Wild Type (WT) mice. Briefly, mice 7 to 8 weeks were sacrificed by cervical dislocation and kidneys were cleaned and rinsed in cold DPBS (Dulbecco's Phosphate-Buffered Saline). The renal cortex was minced and incubated in Dulbecco's modified eagle medium/Nutrient mixture F-12 (DMEM/F12, Sigma) containing 1 mg/mL collagenase A (Roche) during 25 minutes at 37° C. The digestion was inactivated by adding culture medium (DMEM/F12+10% FBS) then the cell suspension was passed through a 100-μm cell strainer. After centrifugation, the pellet (containing MKF) was diluted with culture medium then plated in 75 cm² culture flask. After 24 hours MKF were washed with DPBS before replacement of fresh culture medium. Thereafter, culture medium was changed every 48 hours. Once 70-80% confluent, MKF were trypsinized and plated in 12 or 6-well plates. For experimental use, MKF were starved with culture medium containing only 3% FBS. MKF were treated with mNGAL (500 ng/mL, R&D Systems) for 24 h for gene expression analysis.

### Cell Toxicity and ADME

Two specific toxicity assays have been performed to assess hepatoxicity and cardiotoxicity. Hepatotoxicity was evaluated in isolated hepatocytes from swiss mice and determined by the MTT colorimetric assay. Cardiotoxicity was evaluated using the commercially available Predictor hERG fluorescence polarization kit (Thermo Fisher Scientific). Several ADME assays have been carried out on the compounds generated throughout the project. The following experiments were undertaken: Stability in human microsomes, binding to human plasma protein, and CYP3A4 inhibition assay (Vivid CYP3A4 assay, Thermo Fisher Scientific). Physico-chemical properties were also assessed such as aqueous solubility and chemical stability.

### Cell Permeability

The Caco-2 Permeability assay uses an established method for predicting the in vivo absorption of drugs across the gut wall. The transport rate across the CaCo2 cell line is measured. This cell line has a human colon carcinoma origin and resembles to the intestinal epithelia (a polarised monolayer with microvilli and intercellular tight junctions).

Bidirectional transport (apical to basolateral (A-B) and basolateral to apical (B-A)) across the cell monolayer were determined allowing to calculate the efflux ratio (an indicator as to whether a compound undergoes active efflux).

### Hemodynamics

8-12 weeks old mice (WT C57B16 for GP1 compound experiments) or 8-12 weeks old mice with genetic inactivation of lcn2 (as described in Martinez-martinez et al., Hypertension 2017, December; 70(6):1148-1156) have been used. LV diastolic and systolic diameters were measured in anesthetized (isofluorane 1.5%) mice, according to the American Society of Echocardiography's leading-edge method (using Vivid 7 echograph a 14 Mhz probe). In addition, LV outflow velocity was measured by pulsed waves, and CO was calculated as follows: CO=aortic VTI×[πx·(LV outflow diameter/2)²]×heart rate, where VTI is velocity-time integral.

LV hemodynamic was assessed as described previously.

Mice were anesthetized (chloral 320 mg·kg−1, IP) and the carotid artery cannulated with a pressure-volume catheter (SPR839, Millar-Instruments, USA) and the catheter was advanced into the LV. Pressure-volume loops were obtained at baseline and during loading by gently occluding the abdominal aorta. LV end-systolic and end-diastolic pressures, dP/dt_{max/min}, LV relaxation constant tau and were measured/calculated with IOX software (EMKA, France).

### Blood Pressure (BP)

Systolic BP was measured by tail-cuff plethysmography in trained conscious mice at weeks 8-10 using a BP2000 Visitech model. BP was measured every day in the same room at the same hour for 5 consecutive days. The BP measurements (expressed as mmHg) presented are the averages of the last 3 days

### Chronic Kidney Disease Model

Chronic kidney disease (CKD) was induced by subtotal nephrectomy (Nx) in eight-weeks-old FVB male mice (25-26 g). All surgeries were performed under ketamine/xylazine anaesthesia. Briefly, the left kidney was exposed, and the upper and lower poles were tied with a poly-glycolic acid suture line. The peritoneum and skin were then sutured, and the animals were returned to their individual cages. After one week of recovery, the second kidney was removed. Removal of the second kidney represents TO. Sham mice were subjected to the same surgical procedures but neither renal poles nor the right kidney were removed. Mice were monitored for any sign of distress, and those observed to be experiencing severe, unrelievable pain were euthanized. Renal failure was assessed by the measure of plasma creatinine and urea with an automatic analyser (Konelab 20i; Thermo Fisher Scientific, Vernon Hills, Ill.) at weeks 4 and 10 post-Nx.

### Induction of Salt-Sensitive Hypertension

Salt-sensitive hypertension was induced by co-administration of L-NAME 70 mg/l in drinking water together with 8% NaCl in chow food (High Salt Diet, HSD) for 10 days. GP1 (100 mg/kg/day) was added or not into the food. Control experiments included vehicle (control), L-NAME or high salt (HSD) alone. Systolic blood pressure (expressed as mmHg) was estimated in the last 3 days of treatment.

### Histology and Molecular Biology

After assessment of cardiac hemodynamics, the heart was removed, and the atria and the ventricles were separated and weighed individually. A section of the left ventricle was immersed in Bouin fixative solution. After fixation, the sections were dehydrated and embedded in paraffin. From these sections, 5-m thick histologic slices were obtained and were stained with Sirius Red. For the measurement of cardiac collagen density, slides were examined and photographed under a light microscope (Zeiss) at 40× magnification. Collagen content was calculated as percentage of collagen area to total area of the image. Perivascular collagen was excluded from the analysis.

### Col1 (Col-I) and Col3 (Col-III) Immunostaining

Frozen mid-LV sections are thawed at room temperature (30 min), immersed in acetone, rinsed in PBS (1×) and immersed in BSA (10 min each). Slides are then incubated with primary rabbit anti-human collagen-I Ab (Abcam ab 34710) (dilution 1/1000) and primary goat anti-human collagen-III Ab (Rnd NBP1 26547) (dilution 1/500) for 1 hour. Thereafter, slides are rinsed three times in PBS (1×) before being incubated with secondary antibodies donkey anti-mouse coupled with FITC (Jackson immunosearch 715-095-151) and donkey anti-goat coupled with Cy3 (Interchim A50-201D3) (dilution 1/400). After three rinses in PBS (1×), slides are mounted with a cover slip on a vecatshield+DAPI medium (Vector Laboratories). A minimum of 8/slide microscopic photographs (×20) were taken from Axiocam-Z1 (Zeiss) fluorescence microscope and analyzed with Image Pro-plus Software. Results are shown as mean±esm.

### Molecular Biology

1) Western-Blot Analysis

Total protein aliquots of 20 μg were prepared from cardiac homogenates and electrophoresed on SDS polyacrylamide gels and transferred to Hybond-c Extra nitrocellulose membranes (Amersham Biosciences). Membranes were incubated with primary antibodies for: NGAL (Abcam; dilution 1:500), Collagen type III (Santa Cruz; dilution 1:500), a-SMA (Sigma; dilution 1:1000), CTGF Abcam; dilution 1:500), GDF-15 (Abcam; dilution 1:500), fibronectin (Millipore, 1:500), Galectin-3 (Thermo, 1:1000), Cardiotrophin-1 (Abcam, 1:500), osteopontin (Santa Cruz, 1:1000), cd3 (Abcam, 1:500), cd68 (Abcam, 1:500), cd80 (Santa Cruz, 1:500) and β-actin (Sigma; dilution 1:1000) as a loading control. After washing, the blots were incubated with peroxidase-conjugated secondary antibody, and binding revealed by ECL chemiluminescence (Amersham). After densitometric analyses, optical density values were expressed as arbitrary units. Results are expressed as an n-fold increase over the values of the control group in densitometric arbitrary units.

2) ELISA

NGAL, IL6 and collagen type I concentrations were measured in cardiac tissue and cell supernatants, respectively, by ELISA according to the manufacturer's instructions (R&D Systems).

3) Proliferation

Cell proliferation was assessed using the MTT Proliferation Assay (Sigma).

4) Gene Expression Analysis

Frozen tissues (kidneys, heart) were homogenized in TRIzol (Life Technologies, 15596018) using FastPrep beads (MP-Bio, 6913-100). cDNAs were generated using the Superscript II reverse transcriptase kit (Invitrogen, 18064022), and qPCR was performed as previously described. Briefly, transcript levels were analysed in a CFX396 apparatus (Biorad). The reactions were performed in duplicate for each sample using the IQ SYBR Green supermix Kit (Biorad, 170-8882). To normalize gene expression, the geometric mean of multiple internal reference genes were used (RS16, Ubc, Hprt and Gapdh for mice experiments). Values in control conditions were set as 1 for each gene. The sequences of the specific primers are detailed in Table A.

TABLE A

| Primers | Forward | Reverse |
|---|---|---|
| Target genes | | |
| Collagen I | CCCCGGGACTCCTGGACTT | GCTCCGACACGCCCTCTCTC |
| Fibronectin | CCTACGGCCACTGTGTCACC | AGTCTGGGTCACGGCTGTCT |
| αSMA | TGTGCTGGACTCTGGAGATG | GAAGGAATAGCCACGCTCAG |
| IL6 | CTCTGGGAAATCGTGGAAAT | AAGTGCATCATCGTTGTTCATAC |
| TNFα | G | A |
| MCP1 | GGGACAGTGACCTGGACTGT | AGTGAATTCGGAAAGCCCATT |
| | ACAGGAGAAGGGACGCCAT | GAAGCCCTACAGACGAGCTCA |
| | ATCCCAATGAGTAGGCTGGA | CAGAAGTGCTTGAGGTGGTTGT |
| | GAGC (SEQ ID NO: 1) | G (SEQ ID NO: 2) |

### Statistics

Data are presented as the means±SEM. Student's t test (2-tailed) was used to compare paired groups of independent samples. ANOVA with Bonferroni adjustment for post-hoc tests was used for multiple comparisons.

### Results

### Inhibitory Activity of the First Generation Compounds

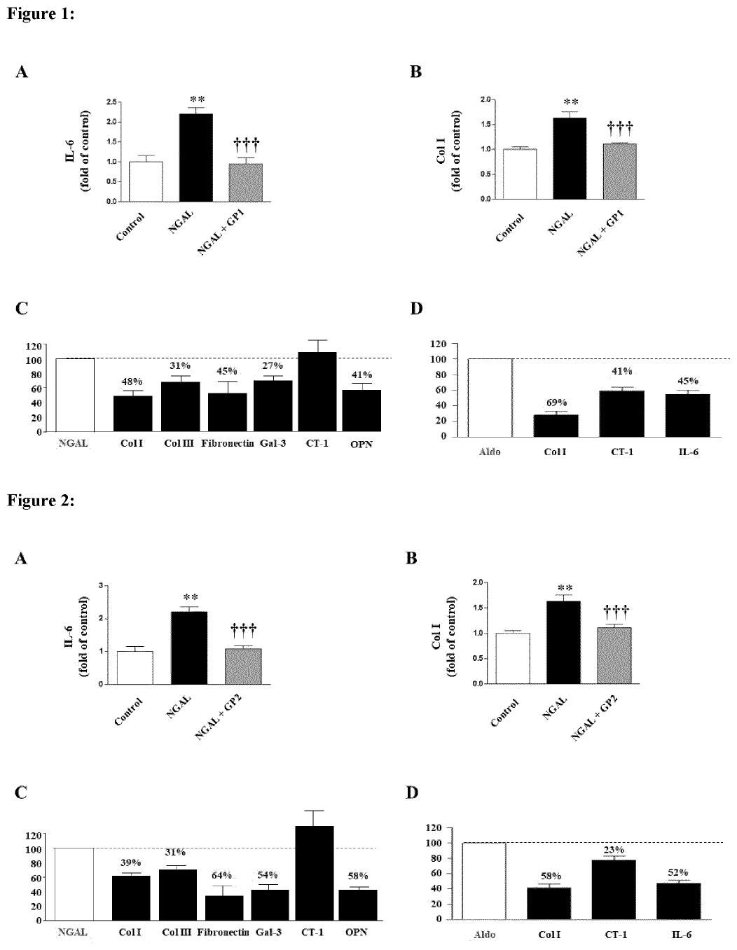

The inhibitory activity of the compounds was evaluated on the NGAL-induced IL6 secretion (NGAL 10 ng/ml) in human cardiac fibroblasts. Among 32 compounds obtained by screening, 2 compounds GPZ614741 and GPZ058225 were selected since they inhibited NGAL-induced IL6 secretion (panel A), Col 1 secretion (panel B), NGAL-induced profibrotic marker (col1, col III, fibronectin, Gal3, OPN) synthesis (panel C) and aldosterone-induced profibrotic marker (Col1, CT-1, or IL6) synthesis (panel D). Results are shown on FIGS. 1 and 2.

TABLE 1

| Compound name | Corresponding chemical formula |
|---|---|
| GPZ058225 (CAS: 519050-14-7) | 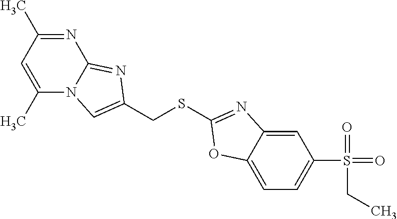 [SMILES: CCS(=O)(=O)c1ccc2oc(SCc3cn4c(C)cc(C)nc4n3)nc2c1] |
| GPZ614741 (CAS: 1241512-52-6) | 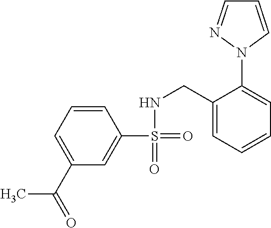 [SMILES: CC(=O)c1cccc(S(=O)(=O)NCc2ccccc2-n2cccn2)c1] |

### Inhibitory Activity of the Second Generation Compound

Inhibitory activities of 23 second generation compounds derived from GPZ614741 and GPZ058225 were evaluated on the NGAL-induced IL6 secretion (NGAL 10 ng/ml) in human cardiac fibroblasts. Results are shown in table 2a and table 2b.

TABLE 2a

| | Corresponding chemical formula |
|---|---|
| GPZ278618 CAS: 1375221-88-7 | 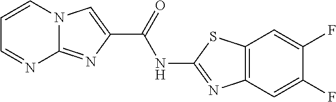 [SMILES: O=C(Nc1nc2cc(F)c(F)cc2s1)c1cn2cccnc2n1] |
| GPZ478519 CAS: 1171056-55-5 | 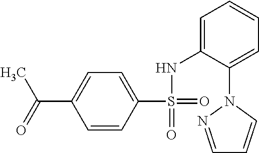 [SMILES: CC(=O)c1ccc(S(=O)(=O)Nc2ccccc2-n2cccn2)cc1] |
| GPZ505884 CAS: 1170422-94-2 | 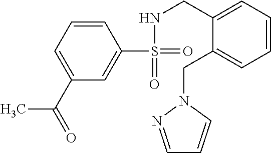 [SMILES: CC(=O)c1cccc(S(=O)(=O)NCc2ccccc2Cn2cccn2)c1] |
| GPZ519431 CAS: 683784-46-5 | 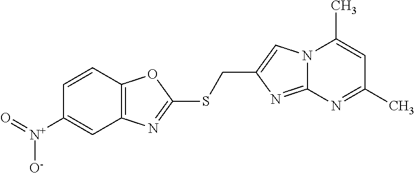 [SMILES: Cc1cc(C)n2cc(CSc3nc4cc([N+](=O)[O-])ccc4o3)nc2n1] |
| GPZ564849 CAS: 300696-62-2 | 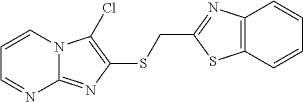 [SMILES: Clc1c(SCc2nc3ccccc3s2)nc2ncccn12] |
| GPZ595600 CAS: 1355610-80-8 | 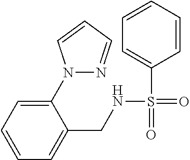 [SMILES: O=S(=O)(NCc1ccccc1-n1cccn1)c1ccccc1] |
| GPZ642292 CAS: 1088151-90-9 | 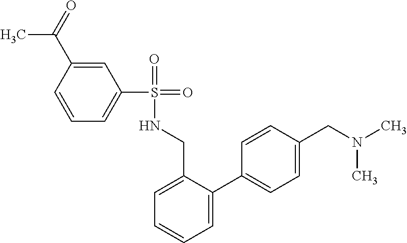 [SMILES: CC(=O)c1cccc(S(=O)(=O)NCc2ccccc2-c2ccc(CN(C)C)cc2)c1] |
| GPZ743042 CAS: 314746-74-2 | 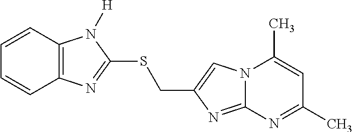 [SMILES: Cc1cc(C)n2cc(CSc3nc4ccccc4[nH]3)nc2n1] |
| GPZ778195 CAS: 1355676-34-4 | 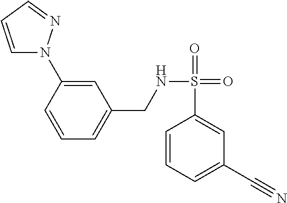 [SMILES: N#Cc1cccc(S(=O)(=O)NCc2cccc(-n3cccn3)c2)c1] |
| GPZ863205 CAS: 1797184-54-3 | 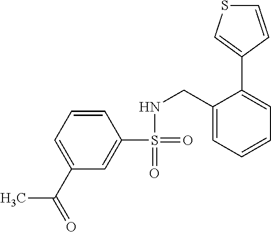 [SMILES: CC(=O)c1cccc(S(=O)(=O)NCc2ccccc2-c2ccsc2)c1] |
| GPZ913629 CAS: 1223268-60-7 | 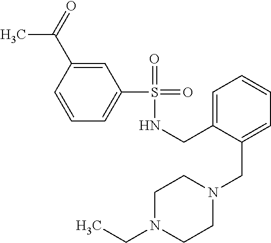 [SMILES: CCN1CCN(Cc2ccccc2CNS(=O)(=O)c2cccc(C(C)=O)c2)CC1] |

TABLE 2b

| Compounds | Activity |
|---|---|
| Control | 1.00 ± 0.03 |
| GPZ278618 | 0.86 ± 0.02 |
| GPZ478519 | 0.69 ± 0.02 |
| GPZ505884 | 0.83 ± 0.02 |
| GPZ519431 | 1.02 ± 0.06 |
| GPZ564849 | 0.85 ± 0.05 |
| GPZ595600 | 0.92 ± 0.04 |
| GPZ642292 | 0.64 ± 0.02 |
| GPZ743042 | 0.94 ± 0.03 |
| GPZ778195 | 0.88 ± 0.04 |
| GPZ863205: | 0.93 ± 0.03 |
| GPZ913629 | 0.93 ± 0.02 |

Identified compounds are as efficient as GPZ614741 and GPZ058225 while other tested compounds have lost their inhibitory activity.

### Drug Repositioning

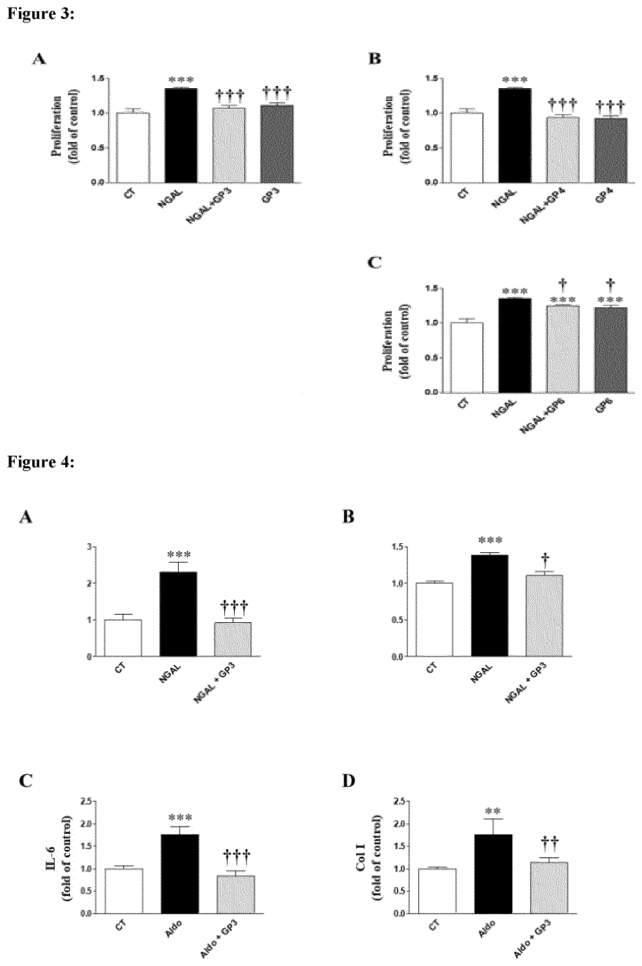

Four candidates selected by virtual screening in the Off-patent Drug database compiled by Greenpharma were evaluated for inhibition of NGAL-induced cell proliferation (NGAL 10 ng/ml) in human cardiac fibroblasts. Results are shown on FIG. 3.

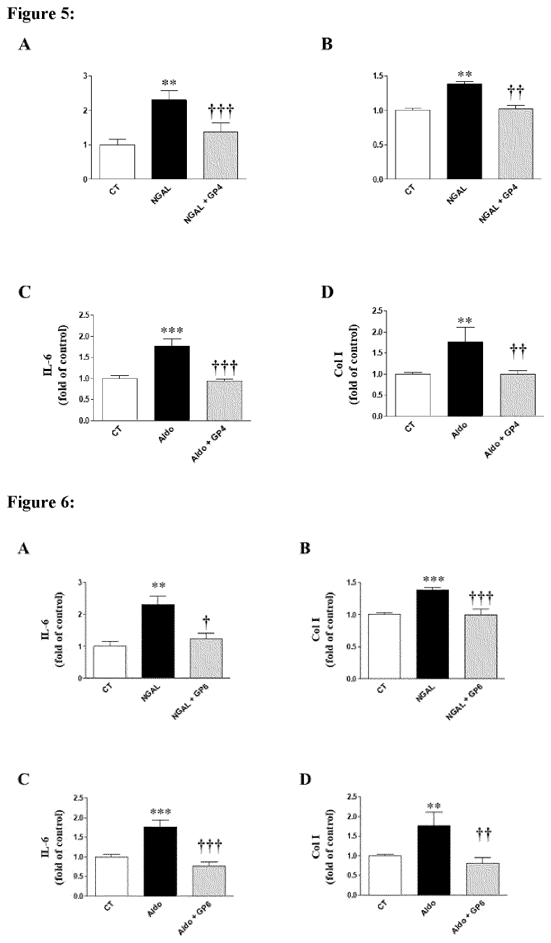

The drug candidates were further evaluated for NGAL- or aldo-modulated inflammatory or profibrotic markers expression. Results are shown on FIGS. 4, 5, and 6.

TABLE 3

| Compound name | Corresponding chemical formula |
|---|---|
| GPZ624624 Sulfaphenazole (CAS: 526-08-9) | 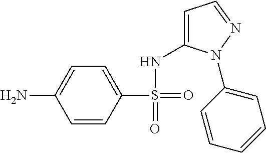 [SMILES: Nc1ccc(S(=O)(=O)Nc2ccnn2-c2ccccc2)cc1] |
| GPZ646083 Imolamine (CAS: 318-23-0) | 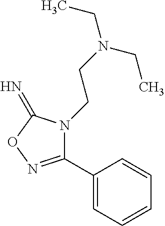 [SMILES: CCN(CC)CCn1c(-c2ccccc2)noc1=N] |
| GPZ706277 Acetohexamide (CAS: 968-81-0) | 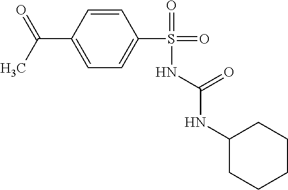 [SMILES: CC(=O)c1ccc(S(=O)(=O)NC(=O)NC2CCCCC2)cc1] |

### Impact of GP1 (GPZ614741) on Cardiac Function in the Myocardial Infarction Model

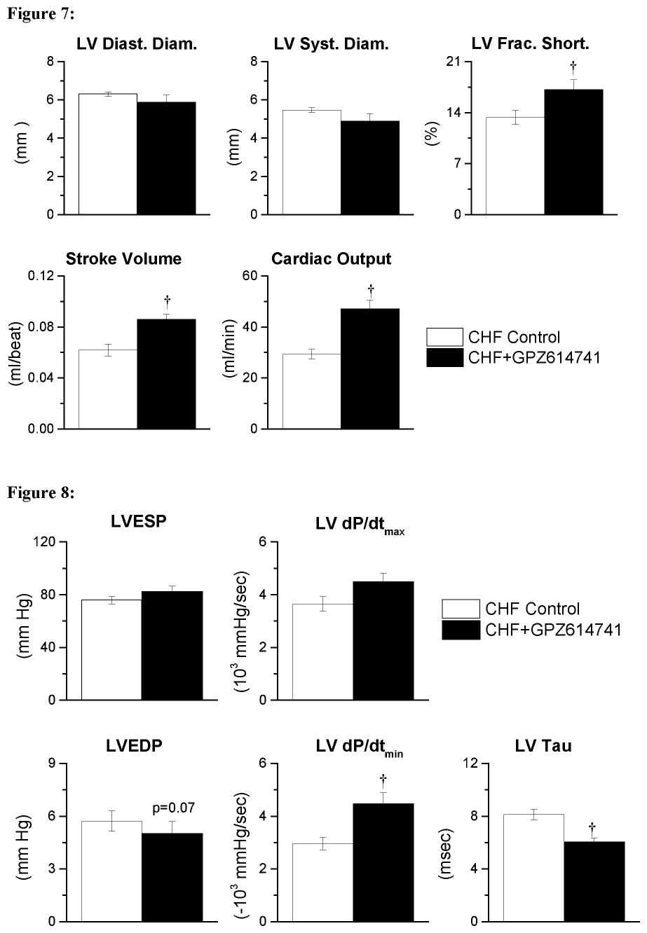

Results are shown on FIGS. 7 and 8.

TABLE 4

| Compound name | Corresponding chemical formula |
|---|---|
| GPZ614741 (CAS: 1241512-52-6) | 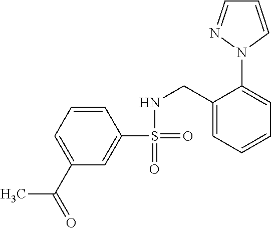 [SMILES: CC(=O)c1cccc(S(=O)(=O)NCc2ccccc2-n2cccn2)c1] |

As shown on FIG. 7, Three months of treatment with GP1 (GPZ614741) (100 mg/kg/day in food admix) resulted in a significant increase in fractional shortening (LV Frac. Short.) (panel A), resulting from a slight although statistical non-significant decrease in both LV diastolic and systolic diameters (LV Diast. Diam. and LV Syst. Diam., respectively (panels B-C). At the same time point, both stroke volume (panel D) and cardiac output (panel E) were increased. This mimics the impact of global genetic inactivation of Lcn2 in the same MI model as reported previously in Martinez-Martinez et al, Hypertension 2017, December; 70(6):1148-1156.

As shown on FIG. 8, Three months of treatment with GP1 (100 mg/kg/day in food admix) resulted in a significant increase in LV dP/dtₘₐₓ ((panel B) while LV end-systolic pressure (LVESP) (panel A) was not modified. Moreover, LV end-diastolic pressure (LVEDP) (panel C) tended to be reduced, but LV dP/dtₘᵢₙ (panel D) was increased and LV relaxation constant Tau was decreased (panel E). This mimics the impact of global genetic inactivation of Lcn2 in the same MI model as reported previously in Martinez-Martinez et al, Hypertension 2017, December; 70(6):1148-1156.

### Impact on Cardiac Fibrosis and Collagen Immunostaining in the Myocardial Infarction Model

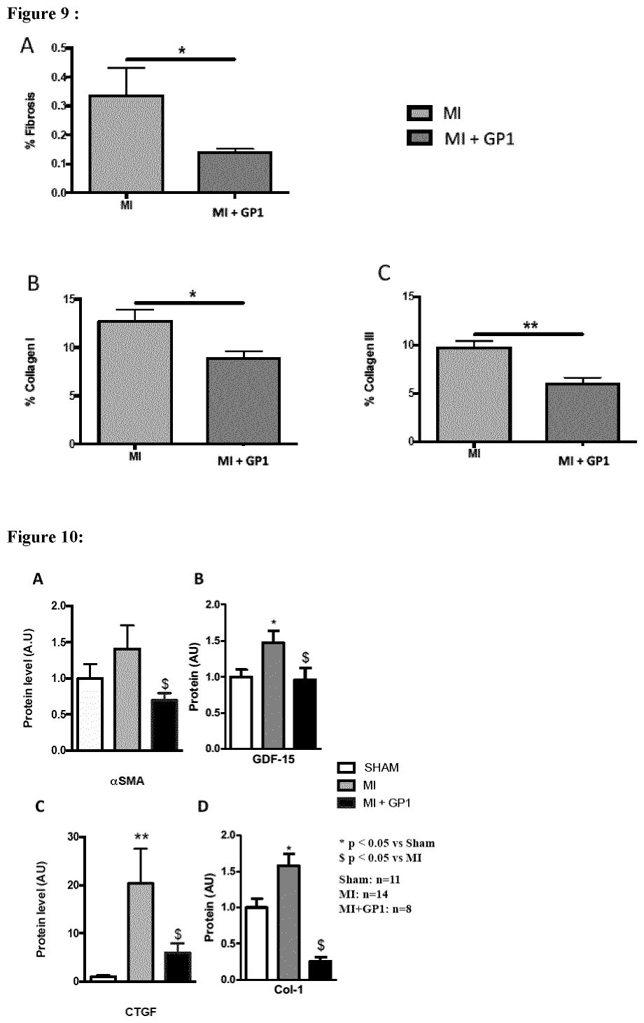

Results are shown on FIG. 9.

As shown on FIG. 9, three months of treatment with GP1 (100 mg/kg/day in food admix) resulted in a significant decrease in LV interstitial collagen density (panel A). Together with a decrease in Col I (panel B) and Col III (panel C) immunostaining. This mimics the impact of global genetic inactivation of Lcn2 in the same MI model as reported previously in Martinez-Martinez et al, Hypertension 2017, December; 70(6):1148-1156.

### Impact on Profibrotic Target Expression in the Myocardial Infarction Model

Results are shown on FIG. 10.

As shown on FIG. 10, three months of treatment with GP1 (100 mg/kg/day in food admix) resulted in significant prevention of up regulation of aSMA (panel A), GDF15 (panel B), CTGF (panel C) or Col1 (panel D) tissue contents. This mimics the impact of global genetic inactivation of Lcn2 in the same MI model as reported previously in Martinez-Martinez et al, Hypertension 2017, December; 70(6):1148-1156).

### Impact on IL6 Expression in the Myocardial Infarction Model

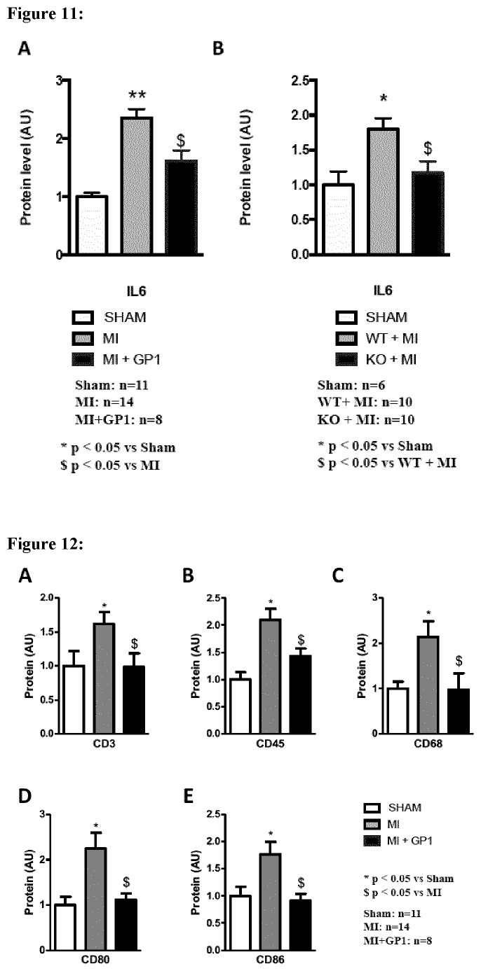

Results are shown on FIGS. 11 and 12.

As shown on FIG. 11, three months of treatment with GP1 (100 mg/kg/day in food admix) resulted in a significant prevention of up regulation of IL6 tissue content (panel A). This mimics the impact of global genetic inactivation of Lcn2 in the same MI model (panel B).

As shown on FIG. 12, three months of treatment with GP1 (100 mg/kg/day in food admix) resulted in a significant prevention of up regulation of CD3 (panel A), CD3 (panel B), CD68 (panel C) CD 80 (panel D) and CD86 (panel E) tissue content. This mimics the impact of global genetic inactivation of Lcn2 in the same MI model as reported previously in Martinez-Martinez et al, Hypertension 2017, December; 70(6):1148-1156).

### Cell Toxicity and ADME

ADME-Toxicity panel showed that GPZ614741 showed no toxicity for hepatocytes (MTT up to 0.1 mM) nor cardiomyocytes (hERG predictor assay). The PPB was low and fully stable to acidic pH. It was also perfectly soluble in aqueous buffers and partially processed by CYP3A4. On the other hand, it was very sensitive to microsomes degradation (2.2% left after 2 h). The ADME-Toxicity panel showed that GPZ058225 had little hepatotoxicity (>0.1 mM) or cardiotoxicity (it did not block hERG channel). Regarding its stability, GPZ058225 was very stable to degradation by microsomes and to digestive content. It also bound to plasma proteins within acceptance range. It had mild solubility problems (only 63% in the solubility test) and CYP3A4 isoform seemed to be involved in its processing (inhibited the enzyme activity in a competition assay by 32%).

TABLE B
Cell toxicity and ADME. 1) Hepato- and cardio-toxicity determination; 2) ADME
drug incompatibility test (CYP3A4 potential); 3) ADME parameters determination.

| | | | ADME | | | | |
|---|---|---|---|---|---|---|---|
| | TOXICITY | | | | | | CyP 3A4 |
| | | Cardiotoxicity | Aquous | Chemical | Micro | | Inhibition % |
| | Hepatotoxicity | % (100 = | solubility | Stability | Stability | hPPB | (100 = no |
| Compounds | μM | no toxicity) | % | % | % | % | inhibition) |
| GP7058225 | No tox up | 101.73 ± 4.1 | 63.46 | 92.2 | 100 | 89.11 | 68.07 ± 1.52 |
| | 100 uM | | | | | | |
| GPZ614741 | No tox up | 101.76 ± 2.48 | 100 | 103 | 2.19 | 71.16 | 72.21 ± 1.94 |
| | 100 uM | | | | | | |

### Cell Permeability

The Bi—CaCo2 assay showed that permeability of GPZ058225 through the intestinal epithelia was quite high (equivalent to the reference compound used).

TABLE C
Bi-CaCo permeability assay.

| | Papp (A-B) | Papp (B-A) | |
|---|---|---|---|
| Compound | cm/sec | cm/sec | Ratio |
| GP7058225 | 3.22 10-5 | 6.81 10-5 | 2.11 |
| propanolol | 2.74 10-5 | 4.28 10-5 | 1.56 |

GPZ058225 showed permeability values in the range of the reference compound (propranolol) used for comparison.

### Impact of GP1 (GPZ614741) on Blood Pressure in the CKD Mouse Model

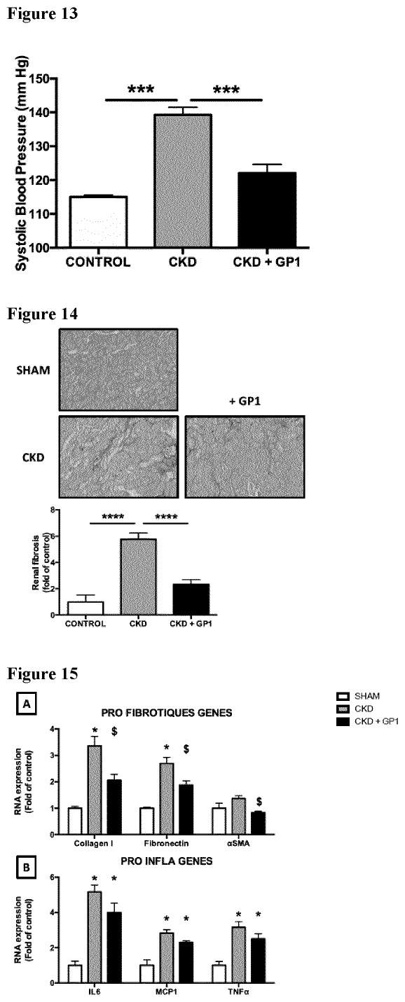

Results are shown on FIG. 13.

As shown in FIG. 13, two months GPZ614741 administration prevented the increase of blood pressure induced by CKD (see FIG. 13).

### Impact of GP1 (GPZ614741) on Renal Fibrosis in the CKD Mouse Model

Results are shown on FIG. 14.

As shown in FIG. 14, two months GP1 (GPZ614741) administration has a strong antifibrotic effect in vivo, blunting the interstitial fibrosis associated to CKD (estimated by sirius red staining) (see FIG. 14 A-B).

### Impact of GP1 (GPZ614741) on Renal Profibrotic and Inflammatory Markers in the CKD Mouse Model

Results are shown on FIG. 15.

As shown in FIG. 15, two months GP1 (GPZ614741) administration blunted the increase of profibrotic target genes (collagen1, fibronectin, alphaSMA) (see FIG. 15A) while it as no effects on inflammatory markers (IL6, MCP1, TNFalpha) (see FIG. 15B).

### Impact of GP1 (GPZ614741) on NGAL-Induced Expression of Profibrotic and Proinflammatory Markers in Renal Fibroblasts

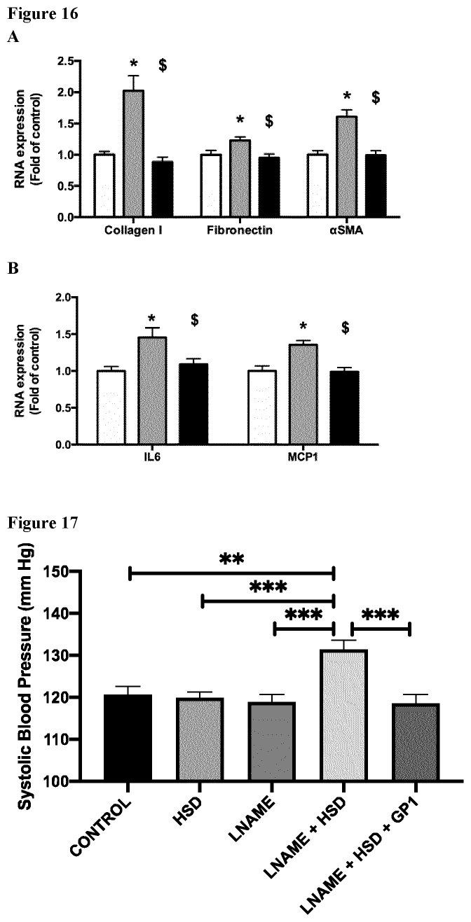

Results are shown on FIG. 16.

As shown on FIG. 16, GP1 (GPZ614741) inhibited the NGAL-induction of profibrotic (Collagen1, fibronectin, aSMA) (see FIG. 16A) or inflammatory (IL6, MCP1) gene expression in renal fibroblast cells stimulated with recombinant murine NGAL in the presence or not of GP1 (GPZ614741).

### Impact of GP1 (GPZ614741) on Salt Induced Hypertension

Results are shown on FIG. 17.

As shown on FIG. 17, GPZ614741 administration prevented the increase of blood pressure induced by the combination of L-NAME and high salt (HSD). Of note L-NAME alone or HSD alone have no impact on blood pressure as compared to vehicle (control).

1. A method of inhibiting NGAL in a subject in need thereof, comprising administering to the subject a therapeutically effective amount of a compound of general formula (I): 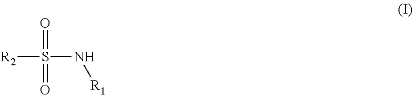
wherein:
R₁ represents:
(CH₂)ₙ₁-pyrazole optionally substituted by an aryl group or a pyridinyl group,
wherein n₁ represent an integer between 0 and 1,
(CH₂)ₙ₂-aryl, said aryl being optionally substituted by one or more:
pyrazolyl groups,
—CH₂-pyrazolyl groups,
thiophenyl groups,
pyridinyl groups or
—CH₂-piperazinyl groups optionally substituted by one or more ethyl groups,
phenyl groups optionally substituted by one or more —(CH₂)n₃-N(—CH₃)(—CH₃),
wherein n₂ and n₃ each independently represent an integer between 0 and 1, or
—C(═O)—N(H)—R₃ wherein R₃ represents a cyclohexyl group;
R₂ represents an aryl group optionally substituted by one or more:
—C(═O)—R₄ wherein R₄ represents a methyl group;
—C≡N; or
—NH₂.

2. The method according to claim 1, wherein:
R₁ represents:
(CH₂)ₙ₂-aryl, said aryl being optionally substituted by one or more:
pyrazolyl groups,
—CH₂-pyrazolyl groups,
thiophenyl groups,
pyridinyl groups, or
—CH₂-piperazinyl groups optionally substituted by one or more ethyl groups,
phenyl groups optionally substituted by one or more —(CH₂)n₃-N(—CH₃)(—CH₃),
wherein n₂ and n₃ each independently represent an integer between 0 and 1;
R₂ represents an aryl group optionally substituted by one or more:
—C(═O)—R₄ wherein R₄ represents a methyl group; or
—C≡N.

3. A method of inhibiting NGAL in a subject in need thereof, comprising administering to the subject a therapeutically effective amount of a compound of general formula (II): 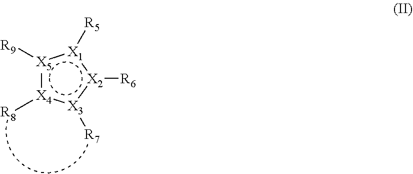
wherein:
X₁ represents a nitrogen atom, or a carbon atom;
X₂ represents a carbon atom, or a CH group;
X₃ represents a nitrogen atom;
X₄ represents an oxygen atom, or a carbon atom;
X₅ represents a carbon atom, or a nitrogen atom;
R₅ represents:
(CH₂)n₄-N(—C₂H₅)(—C₂H₅),
wherein n₄ represents an integer between 0 and 2,
—CH₂—S—R₁₀,
—S—CH₂—R₁₁, or
—C(═O)—N(H)—R₁₂,
wherein R₁₀ R₁₁ and R₁₂ each independently represent an heterocycle of general formula (IV) 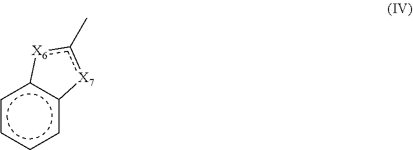
wherein
X₆ represents a nitrogen atom, a —NH group, an oxygen atom, or a sulphur atom,
X₇ represents an oxygen atom or a nitrogen atom,
said heterocycle being optionally substituted by one or more —S(═O)(═O)(—CH₂H₅), —C(═O)Me, halogeno atoms, trifluoromethyl groups, cyano groups, or nitro groups;
R₆ represents a phenyl group, a lone pair, an oxygen atom, or an halogeno atom;
R₉ represents a ═NH group, or a lone pair;
R₇ and R₈ represent a lone pair or R₈—X₄—X₃—R₇ optionally form a six membered ring heterocycle, said heterocycle being optionally substituted by one or more methyl groups, preferably by two methyl groups.

4. The method according to claim 3, wherein the compound is a compound of general formula (III): 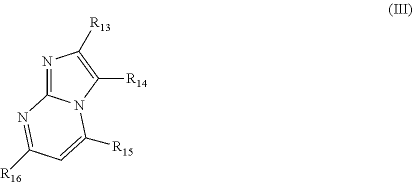
wherein
R₁₃ represents
—CH₂—S—R₁₇,
—S—CH₂—R₁₈, or
—C(═O)—N(H)—R₁₉,
wherein R₁₇, R₁₈ and R₁₉ each independently represent an heterocycle of general formula (V) 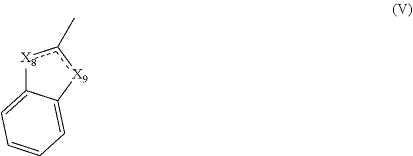
wherein
X₈ represents a nitrogen atom, a —NH group, an oxygen atom, or a sulphur atom,
X₉ represents an oxygen atom or a nitrogen atom,
said heterocycle being optionally substituted by one or more —S(═O)(═O) (—CH₂H₅), halogeno atoms or nitro groups;
R₁₄ represents a lone pair, a hydrogen atom or an halogen atom;
R₁₅ represents a methyl group, a hydrogen atom or a lone pair; and
R₁₆ represents a methyl group, a hydrogen atom or a lone pair.

5-8. (canceled)

9. A method of treating a wound and/or delayed wound closure in a subject in need thereof, comprising
administering to the subject a therapeutically effective amount of
i) the compound of general formula (I): 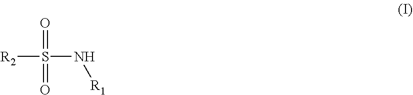
wherein:
R₁ represents:
(CH₂)ₙ₁-pyrazole optionally substituted by an aryl group or a pyridinyl group,
wherein n₁ represent an integer between 0 and 1,
(CH₂)ₙ₂-aryl, said aryl being optionally substituted by one or more:
pyrazolyl groups,
—CH₂-pyrazolyl groups,
thiophenyl groups,
pyridinyl groups or
—CH₂-piperazinyl groups optionally substituted by one or more ethyl groups,
phenyl groups optionally substituted by one or more —(CH₂)n₃-N(—CH₃)(—CH₃),
wherein n₂ and n₃ each independently represent an integer between 0 and 1, or
—C(═O)—N(H)—R₃ wherein R₃ represents a cyclohexyl group;
R₂ represents an aryl group optionally substituted by one or more:
—C(═O)—R₄ wherein R₄ represents a methyl group;
—C≡N; or
—NH₂;
or
ii) the compound of general formula (II): 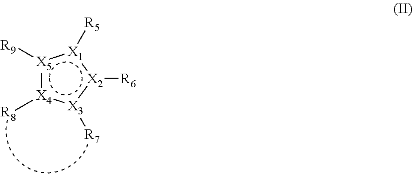
wherein:
X₁ represents a nitrogen atom, or a carbon atom;
X₂ represents a carbon atom, or a CH group;
X₃ represents a nitrogen atom;
X₄ represents an oxygen atom, or a carbon atom;
X₅ represents a carbon atom, or a nitrogen atom;
R₅ represents:
(CH₂)n₄-N(—C₂H₅)(—C₂H₅),
wherein n₄ represents an integer between 0 and 2,
—CH₂—S—R₁₀,
—S—CH₂—R₁₁, or
—C(═O)—N(H)—R₁₂,
wherein R₁₀ R₁₁ and R₁₂ each independently represent an heterocycle of general formula (IV) 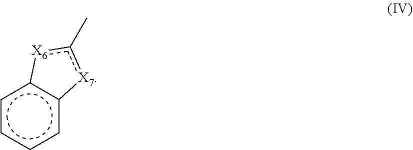
wherein
X₆ represents a nitrogen atom, a —NH group, an oxygen atom, or a sulphur atom,
X₇ represents an oxygen atom or a nitrogen atom,
said heterocycle being optionally substituted by one or more —S(═O)(═O)(—CH₂H₅), —C(═O)Me, halogeno atoms, trifluoromethyl groups, cyano groups, or nitro groups;
R₆ represents a phenyl group, a lone pair, an oxygen atom, or an halogeno atom;
R₉ represents a ═NH group, or a lone pair;
R₇ and R₈ represent a lone pair or R₈—X₄—X₃—R₇ optionally form a six membered ring heterocycle, said heterocycle being optionally substituted by one or more methyl groups, preferably by two methyl groups.

10. A method for treating an NGAL induced disease in a patient in need thereof comprising, administering to the patient a therapeutically effective amount of
i) the compound of general formula (I): 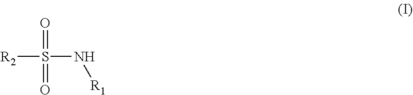
wherein:
R₁ represents:
(CH₂)ₙ₁-pyrazole optionally substituted by an aryl group or a pyridinyl group, wherein n₁ represent an integer between 0 and 1,
(CH₂)ₙ₂-aryl, said aryl being optionally substituted by one or more:
pyrazolyl groups,
—CH₂-pyrazolyl groups,
thiophenyl groups,
pyridinyl groups or
—CH₂-piperazinyl groups optionally substituted by one or more ethyl groups,
phenyl groups optionally substituted by one or more —(CH₂)n₃-N(—CH₃)(—CH₃),
wherein n₂ and n₃ each independently represent an integer between 0 and 1, or
—C(═O)—N(H)—R₃ wherein R₃ represents a cyclohexyl group;
R₂ represents an aryl group optionally substituted by one or more:
—C(═O)—R₄ wherein R₄ represents a methyl group;
—C≡N; or
—NH₂;
or
ii) the compound of general formula (II): 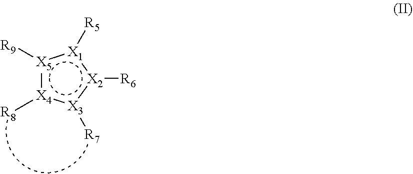
wherein:
X₁ represents a nitrogen atom, or a carbon atom;
X₂ represents a carbon atom, or a CH group;
X₃ represents a nitrogen atom;
X₄ represents an oxygen atom, or a carbon atom;
X₅ represents a carbon atom, or a nitrogen atom;
R₅ represents:
(CH₂)n₄-N(—C₂H₅)(—C₂H₅),
wherein n₄ represents an integer between 0 and 2,
—CH₂—S—R₁₀,
—S—CH₂—R₁₁, or
—C(═O)—N(H)—R₁₂,
wherein R₁₀ R₁₁ and R₁₂ each independently represent an heterocycle of general formula (IV) 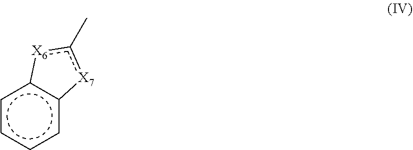
wherein
X₆ represents a nitrogen atom, a —NH group, an oxygen atom, or a sulphur atom,
X₇ represents an oxygen atom or a nitrogen atom,
said heterocycle being optionally substituted by one or more —S(═O)(═O)(—CH₂H₅), —C(═O)Me, halogeno atoms, trifluoromethyl groups, cyano groups, or nitro groups;
R₆ represents a phenyl group, a lone pair, an oxygen atom, or an halogeno atom;
R₉ represents a ═NH group, or a lone pair;
R₇ and R₈ represent a lone pair or R₈—X₄—X₃—R₇ optionally form a six membered ring heterocycle, said heterocycle being optionally substituted by one or more methyl groups, preferably by two methyl groups.

11. The method of claim 9, wherein the wound is a chronic wound.


SEQUENCE LISTING (2 sequences)

SEQ ID NO: 1 - DNA, 143 bp, Artificial Sequence
  Synthetic primer
ccccgggactcctggacttcctacggccactgtgtcacctgtgctggactctggagatgctctgggaaatcgtggaaatggggacagtgacctggactgtacaggagaagggacgccatatcccaatgagtaggctggagagc

SEQ ID NO: 2 - DNA, 149 bp, Artificial Sequence
  Synthetic primer
gctccgacacgccctctctcagtctgggtcacggctgtctgaaggaatagccacgctcagaagtgcatcatcgttgttcatacaagtgaattcggaaagcccattgaagccctacagacgagctcacagaagtgcttgaggtggttgtg